SEQUENCE LISTING (86 sequences)

SEQ ID NO: 1 - protein, 22 aa, Artificial Sequence
  Chimeric
RASGKPVNHSTRKEEKQRNGTL

SEQ ID NO: 2 - protein, 9 aa, Artificial Sequence
  Chimeric
GTRDWIEGE

SEQ ID NO: 3 - protein, 19 aa, Artificial Sequence
  Chimeric
PHLPRALMRSTTKTSGPRA

SEQ ID NO: 4 - protein, 11 aa, Artificial Sequence
  Chimeric
PEWPGSRDKRT

SEQ ID NO: 5 - protein, 5 aa, Artificial Sequence
  Chimeric
EQKDE

SEQ ID NO: 6 - protein, 19 aa, Artificial Sequence
  Chimeric
LSRPSPFDLFIRKSPTITC

SEQ ID NO: 7 - protein, 21 aa, Artificial Sequence
  Chimeric
WLHNEVQLPDARHSTTQPRKT

SEQ ID NO: 8 - protein, 23 aa, Artificial Sequence
  Chimeric
CRASGKPVNHSTRKEEKQRNGLL

SEQ ID NO: 9 - protein, 11 aa, Artificial Sequence
  Chimeric
GKPVNHSTGGC

SEQ ID NO: 10 - protein, 18 aa, Artificial Sequence
  Chimeric
GKPVNHSTRKEEKQRNGC

SEQ ID NO: 11 - protein, 20 aa, Artificial Sequence
  Chimeric
CGKPVNHSTRKEEKQRNGLL

SEQ ID NO: 12 - protein, 14 aa, Artificial Sequence
  Chimeric
RASGKPVNHSTGGC

SEQ ID NO: 13 - protein, 11 aa, Artificial Sequence
  Chimeric
CGTRDWIEGLL

SEQ ID NO: 14 - protein, 12 aa, Artificial Sequence
  Chimeric
CGTRDWIEGETL

SEQ ID NO: 15 - protein, 12 aa, Artificial Sequence
  Chimeric
GTRDWIEGETGC

SEQ ID NO: 16 - protein, 12 aa, Artificial Sequence
  Chimeric
CHPHLPRALMLL

SEQ ID NO: 17 - protein, 12 aa, Artificial Sequence
  Chimeric
CGTHPHLPRALM

SEQ ID NO: 18 - protein, 13 aa, Artificial Sequence
  Chimeric
THPHLPRALMRSC

SEQ ID NO: 19 - protein, 14 aa, Artificial Sequence
  Chimeric
GPHLPRALMRSSSC

SEQ ID NO: 20 - protein, 13 aa, Artificial Sequence
  Chimeric
APEWPGSRDKRTC

SEQ ID NO: 21 - protein, 17 aa, Artificial Sequence
  Chimeric
APEWPGSRDKRTLAGGC

SEQ ID NO: 22 - protein, 17 aa, Artificial Sequence
  Chimeric
CGGATPEWPGSRDKRTL

SEQ ID NO: 23 - protein, 13 aa, Artificial Sequence
  Chimeric
CTRKDRSGPWEPA

SEQ ID NO: 24 - protein, 15 aa, Artificial Sequence
  Chimeric
APCWPGSRDCRTLAG

SEQ ID NO: 25 - protein, 16 aa, Artificial Sequence
  Chimeric
ACPEWPGSRDRCTLAG

SEQ ID NO: 26 - protein, 17 aa, Artificial Sequence
  Chimeric
CATPEWPGSRDKRTLCG

SEQ ID NO: 27 - protein, 16 aa, Artificial Sequence
  Chimeric
CATPEWPGSRDKRTCG

SEQ ID NO: 28 - protein, 13 aa, Artificial Sequence
  Chimeric
TPCWPGSRDKRCG

SEQ ID NO: 29 - protein, 11 aa, Artificial Sequence
  Chimeric
CGAEWEQKDEL

SEQ ID NO: 30 - protein, 11 aa, Artificial Sequence
  Chimeric
AEWEQKDEFIC

SEQ ID NO: 31 - protein, 9 aa, Artificial Sequence
  Chimeric
GEQKDEFIC

SEQ ID NO: 32 - protein, 10 aa, Artificial Sequence
  Chimeric
CAEGEQKDEL

SEQ ID NO: 33 - protein, 19 aa, Artificial Sequence
  Chimeric
CSRPSPFDLFIRKSPTITC

SEQ ID NO: 34 - protein, 18 aa, Artificial Sequence
  Chimeric
CSRPSPFDLFIRKSPTIC

SEQ ID NO: 35 - protein, 17 aa, Artificial Sequence
  Chimeric
CSRPSPFDLFIRKSPTC

SEQ ID NO: 36 - protein, 16 aa, Artificial Sequence
  Chimeric
CSRPSPFDLFIRKSPC

SEQ ID NO: 37 - protein, 15 aa, Artificial Sequence
  Chimeric
CRPSPFDLFIRKSPC

SEQ ID NO: 38 - protein, 16 aa, Artificial Sequence
  Chimeric
CRPSPFDLFIRKSPTC

SEQ ID NO: 39 - protein, 17 aa, Artificial Sequence
  Chimeric
CRPSPFDLFIRKSPTIC

SEQ ID NO: 40 - protein, 18 aa, Artificial Sequence
  Chimeric
CRPSPFDLFIRKSPTITC

SEQ ID NO: 41 - protein, 17 aa, Artificial Sequence
  Chimeric
CPSPFDLFIRKSPTITC

SEQ ID NO: 42 - protein, 16 aa, Artificial Sequence
  Chimeric
CPSPFDLFIRKSPTIC

SEQ ID NO: 43 - protein, 15 aa, Artificial Sequence
  Chimeric
CPSPFDLFIRKSPTC

SEQ ID NO: 44 - protein, 14 aa, Artificial Sequence
  Chimeric
CPSPFDLFIRKSPC

SEQ ID NO: 45 - protein, 20 aa, Artificial Sequence
  Chimeric
CYAFATPEWPGSRDKRTLAC

SEQ ID NO: 46 - protein, 19 aa, Artificial Sequence
  Chimeric
CYAFATPEWPGSRDKRTLC

SEQ ID NO: 47 - protein, 18 aa, Artificial Sequence
  Chimeric
CYAFATPEWPGSRDKRTC

SEQ ID NO: 48 - protein, 17 aa, Artificial Sequence
  Chimeric
CYAFATPEWPGSRDKRC

SEQ ID NO: 49 - protein, 16 aa, Artificial Sequence
  Chimeric
CAFATPEWPGSRDKRC

SEQ ID NO: 50 - protein, 17 aa, Artificial Sequence
  Chimeric
CAFATPEWPGSRDKRTC

SEQ ID NO: 51 - protein, 18 aa, Artificial Sequence
  Chimeric
CAFATPEWPGSRDKRTLC

SEQ ID NO: 52 - protein, 19 aa, Artificial Sequence
  Chimeric
CAFATPEWPGSRDKRTLAC

SEQ ID NO: 53 - protein, 18 aa, Artificial Sequence
  Chimeric
CFATPEWPGSRDKRTLAC

SEQ ID NO: 54 - protein, 17 aa, Artificial Sequence
  Chimeric
CFATPEWPGSRDKRTLC

SEQ ID NO: 55 - protein, 16 aa, Artificial Sequence
  Chimeric
CFATPEWPGSRDKRTC

SEQ ID NO: 56 - protein, 15 aa, Artificial Sequence
  Chimeric
CFATPEWPGSRDKRC

SEQ ID NO: 57 - protein, 17 aa, Artificial Sequence
  Chimeric
CTWSRASGKPVNHSTRC

SEQ ID NO: 58 - protein, 16 aa, Artificial Sequence
  Chimeric
CTWSRASGKPVNHSTC

SEQ ID NO: 59 - protein, 15 aa, Artificial Sequence
  Chimeric
CTWSRASGKPVNHSC

SEQ ID NO: 60 - protein, 14 aa, Artificial Sequence
  Chimeric
CTWSRASGKPVNHC

SEQ ID NO: 61 - protein, 13 aa, Artificial Sequence
  Chimeric
CWSRASGKPVNHC

SEQ ID NO: 62 - protein, 14 aa, Artificial Sequence
  Chimeric
CWSRASGKPVNHSC

SEQ ID NO: 63 - protein, 15 aa, Artificial Sequence
  Chimeric
CWSRASGKPVNHSTC

SEQ ID NO: 64 - protein, 16 aa, Artificial Sequence
  Chimeric
CWSRASGKPVNHSTRC

SEQ ID NO: 65 - protein, 15 aa, Artificial Sequence
  Chimeric
CSRASGKPVNHSTRC

SEQ ID NO: 66 - protein, 14 aa, Artificial Sequence
  Chimeric
CSRASGKPVNHSTC

SEQ ID NO: 67 - protein, 13 aa, Artificial Sequence
  Chimeric
CSRASGKPVNHSC

SEQ ID NO: 68 - protein, 12 aa, Artificial Sequence
  Chimeric
CSRASGKPVNHC

SEQ ID NO: 69 - protein, 17 aa, Artificial Sequence
  Chimeric
CQWLHNEVQLPDARHSC

SEQ ID NO: 70 - protein, 16 aa, Artificial Sequence
  Chimeric
CQWLHNEVQLPDARHC

SEQ ID NO: 71 - protein, 15 aa, Artificial Sequence
  Chimeric
CQWLHNEVQLPDARC

SEQ ID NO: 72 - protein, 14 aa, Artificial Sequence
  Chimeric
CQWLHNEVQLPDAC

SEQ ID NO: 73 - protein, 13 aa, Artificial Sequence
  Chimeric
CWLHNEVQLPDAC

SEQ ID NO: 74 - protein, 14 aa, Artificial Sequence
  Chimeric
CWLHNEVQLPDARC

SEQ ID NO: 75 - protein, 15 aa, Artificial Sequence
  Chimeric
CWLHNEVQLPDARHC

SEQ ID NO: 76 - protein, 16 aa, Artificial Sequence
  Chimeric
CWLHNEVQLPDARHSC

SEQ ID NO: 77 - protein, 15 aa, Artificial Sequence
  Chimeric
CLHNEVQLPDARHSC

SEQ ID NO: 78 - protein, 14 aa, Artificial Sequence
  Chimeric
CLHNEVQLPDARHC

SEQ ID NO: 79 - protein, 13 aa, Artificial Sequence
  Chimeric
CLHNEVQLPDARC

SEQ ID NO: 80 - protein, 12 aa, Artificial Sequence
  Chimeric
CLHNEVQLPDAC

SEQ ID NO: 81 - protein, 6 aa, Artificial Sequence
  Chimeric
LFIRKS

SEQ ID NO: 82 - protein, 7 aa, Artificial Sequence
  Chimeric
PSKGTVN

SEQ ID NO: 83 - protein, 23 aa, Artificial Sequence
  Chimeric
LHNEVQLPDARHSTTQPRKTKGS

SEQ ID NO: 84 - protein, 6 aa, Artificial Sequence
  Chimeric
SVNPGK

SEQ ID NO: 85 - protein, 13 aa, Artificial Sequence
  Chimeric
CPEWPGCRDKRTG

SEQ ID NO: 86 - protein, 13 aa, Artificial Sequence
  Chimeric
TPEWPGCRDKRCG

(19) United States
(12) Patent Application Publication
(10) Pub. No.: US 2003/0147906 A1
(43) Pub. Date: Aug. 7, 2003

(54) Epitopes or mimotopes derived from the C-epsilon-3 or C-epsilon-4 domains of lgE, antagonists thereof, and their therapeutic uses

(75) Inventors: Martin Friede, Cardiff, CA (US); Sean Mason, Cambridge (GB); WIlliam Gordon Turnell, Cambridge (GB); Marcelle Paulette Van Mechelen, Wagnelee (BE); Carlotta Vinals y de Bassols, Brussels (BE)

Correspondence Address: GLAXOSMITHKLINE, Corporate Intellectual Property - UW2220, P.O. Box 1539, King of Prussia, PA, 19406-0939, US

(73) Assignee: SmithKline Beecham Biologicals, s.a.

(21) Appl. No.: 10/322,210

(22) Filed: Dec. 18, 2002

Related U.S. Application Data
(63) Continuation of application No. 09/914,089, filed on Nov. 2, 2001, now abandoned.
Application No. 09/914,089 is a 371 of international application No. PCT/EP00/01456, filed on Feb. 22, 2000.

(30) Foreign Application Priority Data
Jul. 21, 1999 (GB) 9917144.9
Aug. 7, 1999 (GB) 9918598.5
Aug. 7, 1999 (GB) 9918599.3
Aug. 7, 1999 (GB) 9918601.7
Aug. 7, 1999 (GB) 9918604.1
Aug. 7, 1999 (GB) 9918606.6
Oct. 29, 1999 (GB) 9925618.2

(51) Int. Cl. A61K 39/12; C07K 2/00; C07K 4/00; C07K 5/00; C07K 7/00; C07K 14/00; C07K 16/00; C07K 17/00; A61K 38/00; C07K 1/00

(52) U.S. Cl. 424/186.1; 424/204.1; 530/300; 530/350

(57) ABSTRACT

The present invention relates to the provision of novel medicaments for the treatment, prevention or amelioration of allergic disease. In particular, the novel medicaments are epitopes or mimotopes derived from the Cε3 or Cε4 domains of IgE. These novel regions may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the IgE regions of the present invention, and their use in medicine as passive immunotherapy or immunoprophylaxis.

The present invention relates to the provision of novel medicaments for the treatment, prevention or amelioration of allergic disease. In particular, the novel medicaments are epitopes or mimotopes derived from the Cε3 or Cε4 domains of IgE. These novel regions may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the IgE regions of the present invention, and their use in medicine as passive immunotherapy or immunoprophylaxis.

In an allergic response, the symptoms commonly associated with allergy are brought about by the release of allergic mediators, such as histamine, from immune cells into the surrounding tissues and vascular structures. Histamine is normally stored in mast cells and basophils, until such time as the release is triggered by interaction with allergen specific IgE. The role of IgE in the mediation of allergic responses, such as asthma, food allergies, atopic dermatitis, type-I hypersensitivity and allergic rhinitis, is well known. On encountering an antigen, such as pollen or dust mite allergens, B-cells commence the synthesis of allergen specific IgE. The allergen specific IgE then binds to the FcεRI receptor (the high affinity IgE receptor) on basophils and mast cells. Any subsequent encounter with allergen leads to the triggering of histamine release from the mast cells or basophils, by cross-linking of neighbouring IgE/FcεRI complexes (Sutton and Gould, Nature, 1993, 366: 421-428; EP 0 477 231 B1).

IgE, like all immunoglobulins, comprises two heavy and two light chains. The ε heavy chain consists of five domains: one variable domain (VH) and four constant domains (Cε1 to Cε4). The molecular weight of IgE is about 190,000 Da, the heavy chain being approximately 550 amino acids in length. The structure of IgE is discussed in Padlan and Davis (Mol. Immunol., 23, 1063-75, 1986) and Helm et al., (2IgE model structure deposited 2/10/90 with PDB (Protein Data Bank, Research Collabarotory for Structural Bioinformatics; http:\pdb-browsers.ebi.ac.uk)). Each of the IgE domains consists of a squashed barrel of seven anti-parallel strands of extended (β-) polypeptide segments, labelled a to f, grouped into two β-sheets. Four β-strands (a,b, d & e) form one sheet that is stacked against the second sheet of three strands (c,f & g) (see FIG. 8). The shape of each β-sheet is maintained by lateral packing of amino acid residue side-chains from neighbouring anti-parallel strands within each sheet (and is further stabilised by main-chain hydrogen-bonding between these strands). Loops of residues, forming non-extended (non-β-) conformations, connect the anti-parallel β-strands, either within a sheet or between the opposing sheets. The connection from strand a to strand b is labelled as the A-B loop, and so on. The A-B and d-e loops belong topologically to the four-stranded sheet, and loop f-g to the three-stranded sheet. The interface between the pair of opposing sheets provides the hydrophobic interior of the globular domain. This water-inaccessible, mainly hydrophobic core results from the close packing of residue side-chains that face each other from opposing β-sheets.

In the past, a number of passive or active immunotherapeutic approaches designed to interfere with IgE-mediated histamine release mechanism have been investigated. These approaches include interfering with IgE or allergen/IgE complexes binding to the FcεRI or FcεRII (the low affinity IgE receptor) receptors, with either passively administered antibodies, or with passive administration of IgE derived peptides to competitively bind to the receptors. In addition, some authors have described the use of specific peptides derived from IgE in active immunisation to stimulate histamine release inhibiting immune responses.

In the course of their investigations, previous workers in this field have encountered a number of considerations, and problems, which have to be taken into account when designing new anti-allergy therapies. One of the most dangerous problems revolves around the involvement of IgE cross-linking in the histamine release signal. It is most often the case that the generation of anti-IgE antibodies during active vaccination, are capable of triggering histamine release per se, by the cross-linking of neighbouring IgE-receptor complexes in the absence of allergen. This phenomenon is termed anaphylactogenicity. Indeed many commercially available anti-IgE monoclonal antibodies which are normally used for IgE detection assays, are anaphylactogenic, and consequently useless and potentially dangerous if administered to a patient.

Whether or not an antibody is anaphylactogenic, depends on the location of the target epitope on the IgE molecule. However, based on the present state of knowledge in this area, and despite enormous scientific interest and endeavour, there is little or no predictability of what characteristics any antibody or epitope may have and whether or not it might have a positive or negative clinical effect on a patient.

Therefore, in order to be safe and effective, the passively administered, or vaccine induced, antibodies must bind in a region of IgE which is capable of interfering with the histamine triggering pathway, without being anaphylactic per se. The present invention achieves all of these aims and provides medicaments which are capable of raising non-anaphylactic antibodies which inhibit histamine release. These medicaments may form the basis of an active vaccine or be used to raise appropriate antibodies for passive immunotherapy, or may be passively administered themselves for a therapeutic effect.

Much work has been carried out by those skilled in the art to identify specific anti-IgE antibodies which do have some beneficial effects against IgE-mediated allergic reaction (WO 90/15878, WO 89/04834, WO 93/05810). Attempts have also been made to identify epitopes recognised by these useful antibodies, to create peptide mimotopes of such epitopes and to use those as immunogens to produce anti-IgE antibodies.

WO 97/31948 describes an example of this type of work, and further describes IgE peptides from the Cε3 and Cε4 domains conjugated to carrier molecules for active vaccination purposes. These immunogens may be used in vaccination studies and are said to be capable of generating antibodies which subsequently inhibit histamine release in vivo . In this work, a monoclonal antibody (BSW17) was described which was said to be capable of binding to IgE peptides contained within the Cε3 domain which are useful for active vaccination purposes.

EP 0 477 231 B1 describes immunogens derived from the Cε4 domain of IgE (residues 497-506, also known as the Stanworth decapeptide), conjugated to Keyhole Limpet Haemocyanin (KLH) used in active vaccination immunoprophylaxis. WO 96/14333 is a continuation of the work described in EP 0 477 231 B1.

Other approaches are based on the identification of peptides derived from Cε3 or Cε4, which themselves compete for IgE binding to the high or low affinity receptors on basophils or mast cells (WO 93/04173, WO 98/24808, EP 0 303 625 B1, EP 0 341 290).

The present invention is the identification of novel sequences of IgE which are used in active or passive immunoprophylaxis or therapy. These sequences have not previously been associated with anti-allergy treatments. The present invention provides peptides, per se, that incorporate specific isolated epitopes from continuous portions of IgE which have been identified as being surface exposed, and further provides mimotopes of these newly identified epitopes. These peptides or mimotopes may be used alone in the treatment of allergy, or may be used vaccines to induce auto anti-IgE antibodies during active immunoprophylaxis or immunotherapy of allergy to limit, reduce, or eliminate allergic symptoms in vaccinated subjects.

Surprisingly, the anti-IgE antibodies induced by the peptides of the present invention are non-anaphylactogenic and are capable of blocking IgE-mediated histamine release from mast cells and basophils.

The regions of human IgE which are peptides of the present invention, and which may serve to provide the basis for peptide modification are:

TABLE 1

| | | | |
|---|---|---|---|
| | | Location | |
| | | sequence | SEQ |
| | | and IgE | ID |
| Peptide | Sequence | Domain | NO. |

| | | | |
|---|---|---|---|
| P5 | RASGKPVNHSTRKEEKQRNGTL | Cε3 | 1 |
| P6 | GTRDWIEGE | Cε3 | 2 |
| P7 | PHLPRALMRSTTKTSGPRA | Cε3/Cε4 | 3 |
| P8 | PEWPGSRDKRT | Cε4 (Pro451- | 4 |
| | | Thr461) | |
| P9 | EQKDE | Cε4 | 5 |
| P200 | LSRPSPFDLFIRKSPTITC | Cε3 | 6 |
| P210 | WLHNEVQLPDARHSTTQPRKT | Cε4 | 7 |
| 1-90N | LFIRKS | Cε3 | 81 |
| 2-90N | PSKGTVN | Cε3 | 82 |
| 3-90N | LHNEVQLPDARHSTTQPRKTKGS | Cε4 | 83 |
| 4-90N | SVNPGK | Cε4 | 84 |

Peptides that incorporate these epitopes form a preferred aspect of the present invention. Mimotopes which have the same characteristics as these epitopes, and immunogens comprising such mimotopes which generate an immune response which cross-react with the IgE epitope in the context of the IgE molecule, also form part of the present invention.

The present invention, therefore, includes isolated peptides encompassing these IgE epitopes themselves, and any mimotope thereof. The meaning of mimotope is defined as an entity which is sufficiently similar to the native IgE epitope so as to be capable of being recognised by antibodies which recognise the native IgE epitope; (Gheysen, H. M., et al., 1986, Synthetic peptides as antigens. Wiley, Chichester, Ciba foundation symposium 119, p130-149; Gheysen, H. M., 1986, Molecular Immunology, 23,7, 709-715); or are capable of raising antibodies, when coupled to a suitable carrier, which antibodies cross-react with the native IgE epitope.

The mimotopes of the present invention may be peptidic or non-peptidic. A peptidic mimotope of the surface exposed IgE epitopes identified above, may also be of exactly the same sequence as the native epitope. Such a molecule is described as a mimotope of the epitope, because although the two molecules share the same sequence, the mimotope will not be presented in the context of the whole IgE domain structure, and as such the mimotope may take a slightly different conformation to that of the native IgE epitope. It will also be clear to the man skilled in the art that the above identified linear sequences (P1 to P7), when in the tertiary structure of IgE, lie adjacent to other regions that may be distant in the primary sequence of IgE. As such, for example, a mimotope of P1 may be continuous or discontinuous, in that it comprises or mimics segments of P1 and segments made up of these distant amino acid residues.

The mimotopes of the present invention mimic the surface exposed regions of the IgE structure, however, within those regions the dominant aspect is thought by the present inventors to be those regions within the surface exposed area which correlate to a loop structure. The structure of the domains of IgE are described in “Introduction to protein Structure” (page 304, 2^{nd} Edition, Branden and Tooze, Garland Publishing, New York, ISBN 0 8153 2305-0) and take the form a β-barrel made up of two opposing anti-parallel β-sheets (see FIG. 8). The mimotopes may comprise, therefore, a loop with N or C terminal extensions which may be the natural amino acid residues from neighbouring sheets. As examples of this, P100 contains the A-B loop of Cε3, P8 contains the A-B loop of Cε4, P5 contains the C-D loop of Cε3 and P110 contains the C-D loop of Cε4. Accordingly, mimotopes of these loops form an aspect of the present invention. Particularly preferred loops are the C-D loops of Cε3 or Cε4, and the A-B loop of Cε4.

Peptide mimotopes of the above-identified IgE epitopes may be designed for a particular purpose by addition, deletion or substitution of elected amino acids. Thus, the peptides of the present invention may be modified for the purposes of ease of conjugation to a protein carrier. For example, it may be desirable for some chemical conjugation methods to include a terminal cysteine to the IgE epitope. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the IgE peptide as found in the context of the whole IgE molecule. For example, the peptides may be altered to have an N-terminal cysteine and a C-terminal hydrophobic amidated tail. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids. In addition, these may be cyclised by techniques known in the art to constrain the peptide into a conformation that closely resembles its shape when the peptide sequence is in the context of the whole IgE molecule. A preferred method of cyclising a peptide comprises the addition of a pair of cysteine residues to allow the formation of a disulphide bridge.

Further, those skilled in the art will realise that mimotopes or immunogens of the present invention may be larger than the above-identified epitopes, and as such may comprise the sequences disclosed herein. Accordingly, the mimotopes of the present invention may consist of addition of N and/or C terminal extensions of a number of other natural residues at one or both ends. The peptide mimotopes may also be retro sequences of the natural IgE sequences, in that the sequence orientation is reversed; or alternatively the sequences may be entirely or at least in part comprised of D-stereo isomer amino acids (inverso sequences). Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system (for example P14c).

Alternatively, peptide mimotopes may be identified using antibodies which are capable themselves of binding to the IgE epitopes of the present invention using techniques such as phage display technology (EP 0 552 267 B1). This technique, generates a large number of peptide sequences which mimic the structure of the native peptides and are, therefore, capable of binding to anti-native peptide antibodies, but may not necessarily themselves share significant sequence homology to the native IgE peptide. This approach may have significant advantages by allowing the possibility of identifying a peptide with enhanced immunogenic properties (such as higher affinity binding characteristics to the IgE receptors or anti-IgE antibodies, or being capable of inducing polyclonal immune response which binds to IgE with higher affinity), or may overcome any potential self-antigen tolerance problems which may be associated with the use of the native peptide sequence. Additionally this technique allows the identification of a recognition pattern for each native-peptide in terms of its shared chemical properties amongst recognised mimotope sequences.

Examples of such mimotopes are:

TABLE 2

| | | | |
|---|---|---|---|
| | | hl,23 | SEQ |
| | | | ID |
| Peptide | Sequence | Description | SEQ ID NO. |

| | | | |
|---|---|---|---|
| P11 | CRASGKPVNHSTRKEEKQRNGLL | P5 mimotope | 8 |
| P11a | (Ac)GKPVNHSTGGC | P5 mimotope | 9 |
| P11b | (Ac)GKPVNHSTRKEEKQRNGC | P5 mimotope | 10 |
| P11c | CGKPVNHSTRKEEKQRNGLL(NH₂) | P5 mimotope | 11 |
| P11d | (Ac)RASGKPVNHSTGGC | P5 mimotope | 12 |
| P12 | CGTRDWIEGLL | P6 mimotope | 13 |
| P12a | CGTRDWIEGETL(NH₂) | P6 mimotope | 14 |
| P12b | (Ac)GTRDWIEGETGC | P6 mimotope | 15 |
| P13 | CHPHLPRALMLL | P7 mimotope | 16 |
| P13a | CGTHPHLPRALM(NH₂) | P7 mimotope | 17 |
| P13b | (Ac)THPHLPRALMRSC | P7 mimotope | 18 |
| P13c | (Ac)GPHLPRALMRSSSC | P7 mimotope | 19 |
| P14 | APEWPGSRDKRTC | P8 mimotope | 20 |
| P14a | (Ac)APEWPGSRDKRTLAGGC | P8 mimotope | 21 |
| P14b | CGGATPEWPGSRDKRTL(NH₂) | P8 mimotope | 22 |
| P14c | CTRKDRSGPWEPA(NH₂) | P8 retro | 23 |
| P14d* | (Ac)APCWPGSRDCRTLAG | P8 mimotope | 24 |
| (cyclic) | | | |
| P14d | (Ac)ACPEWPGSRDRCTLAG | P8 mimotope | 25 |
| (cyclic) | | | |
| C-1C14 | CATPEWPGSRDKRTLCG | P8 mimotope | 26 |
| C-1C13 | CATPEWPGSRDKRTCG | P8 mimotope | 27 |
| C3C12 | TPCWPGSRDKRCG | P8 mimotope | 28 |
| P9a | CGAEWEQKDEL(NH₂) | P9 mimotope | 29 |
| P9b | (Ac)AEWEQKDEFIC | P9 mimotope | 30 |
| P9b* | (Ac)GEQKDEFIC | P9 mimotope | 31 |
| P9a* | CAEGEQKDEL(NH₂) | P9 mimotope | 32 |
| Carl1 | CPEWPGCRDKRTG | P8 mimotope | 85 |
| Carl2 | TPEWPGCRDKRCG | P8 mimotope | 86 |

Alternatively, peptide mimotopes may be generated with the objective of increasing the immunogenicity of the peptide by increasing its affinity to the anti-IgE peptide polyclonal antibody, the effect of which may be measured by techniques known in the art such as (Biocore experiments). In order to achieve this the peptide sequence may be electively changed following the general rules:

To maintain the structural constraints, prolines and glycines should not be replaced

Other positions can be substituted by an amino acid that has similar physicochemical properties.

As such, each amino acid residue can be replaced by the amino acid that most closely resembles that amino acid. For example, A may be substituted by V, L or I, as described in the following table.

| Original | Exemplary | Preferred |
|---|---|---|
| residue | substitutions | substitution |
| A | V, L, I | V |
| R | K, Q, N | K |
| N | Q, H, K, R | Q |
| D | E | E |
| C | S | S |
| Q | N | N |
| E | D | D |
| G | A | A |
| H | N, Q, K, R | N |
| I | L, V, M, A, F | L |
| L | I, V, M, A, F | I |
| K | R, Q, N | R |
| M | L, F, I | L |
| F | L, V, I, A, Y, W | W |
| P | A | A |
| S | T | T |
| T | S | S |
| W | Y, F | Y |
| Y | W, F, T, S | F |
| V | I, L, M, F, A | L |

Particularly preferred IgE peptides are P8 and variants thereof (such as P14 or P14a). These peptides, when coupled to a carrier are potent in inducing anti-IgE immune responses, which responses are capable of inhibiting histamine release from human basophils. Variants, or mimotopes, of P8 are described primarily as any peptide based immunogen which is capable of inducing an immune response, which response is capable of recognising P8. Without being limiting to the scope of the present invention, some variants of P8 may be described by a general formula in which certain amino acids may be replaced by their closest counterparts. Using this technique, P8 peptide mimotopes may be described by the general formula:

P, X₁, X₂, P, X₃, X₄, X₅, X₆, X₅, X₅

or,

P, X₁, X₂, P, G, X₄, R, D, X₅, X₅

wherein; X₁ is an amino acid selected from E, D, N, or Q; X₂ is an amino acid selected from W, Y, or F; X₃ is an amino acid selected from G or A, X₄ is an amino acid selected from S, T or M; X₅ is an amino acid selected from R or K; and X₆ is an amino acid selected from D or E.

P8 mimotopes may also be identified using antibodies which are capable themselves of binding to P8, using techniques such as phage display technology (EP 0 552 267 B1). Monoclonal antibodies such as P14/23, P14/31 and P14/33 are particularly suitable in this regard.

The present invention, therefore, provides novel epitopes, and mimotopes thereof, and their use in the manufacture of pharmaceutical compositions for the prophylaxis or therapy of allergies. Immunogens comprising at least one of the epitopes or mimotopes of the present invention and carrier molecules are also provided for use in vaccines for the immunoprophylaxis or therapy of allergies. Accordingly, the epitopes, mimotopes, or immunogens of the present invention are provided for use in medicine, and in the medical treatment or prophylaxis of allergic disease. Preferred immunogens and vaccines of the present invention comprise the IgE epitope P8, or mimotopes thereof, including P14.

The present inventors have shown that different methods by which the epitope or mimotope is presented has significant effects upon binding to monoclonal antibodies and to the immune response after vaccination. For example, when using cyclised peptides, altering the length and phase of the loop may have significant effects on the binding activity of the cyclised mimotopes to the P14 monoclonal antibodies (P14/23, P14/31 or P14/33). As such the present inventors have developed a novel system which selects the sites of cyclisation, thereby increasing the probability that the cyclised peptides are held in the correct loop structure, which comprises the correct amino acid residues. In this way, the peptide is likely to be constrained in a conformation that most closely resembles that which the peptides would normally adopt if they were in the context of the whole IgE domain. Hence, without limiting the present invention the cyclised mimotopes which follow these new rules form one preferred aspect of the present invention.

Putative mimotope sequences that are not consistent with these rules may still raise useful antisera (for example P14 and P11), as such the following examples are only a sub-set of the types of mimotopes of the present invention.

Examples of preferred peptides that follow these newly defined structural rules are:

TABLE 3

| Peptide sequence | Mimotope of | SEQ ID NO. |
|---|---|---|
| CSRPSPFDLFIRKSPTITC | A-B loop of Cε3 | 33 |
| CSRPSPFDLFIRKSPTC | A-B loop of Cε3 | 35 |
| CPSPFDLFIRKSPTITC | A-B loop of Cε3 | 41 |
| CPSPFDLFIRKSPC | A-B loop of Cε3 | 43 |
| CTWSRASGKPVNHSTC | C-D loop of Cε3 | 58 |
| CTWSRASGKPVNHC | C-D loop of Cε3 | 60 |
| CSRASGKPVNHSTC | C-D loop of Cε3 | 66 |
| CSRASGKPVNHC | C-D loop of Cε3 | 68 |
| CYAFATPEWPGSRDKRTLAC | A-B loop of Cε4 | 45 |
| CYAFATPEWPGSRDKRTC | A-B loop of Cε4 | 47 |
| CFATPEWPGSRDKRTLAC | A-B loop of Cε4 | 53 |
| CFATPEWPGSRDKRTC | A-B loop of Cε4 | 55 |
| CQWLHNEVQLPDARHC | C-D loop of Cε4 | 70 |
| CQWLHNEVQLPDAC | C-D loop of Cε4 | 72 |
| CLHNEVQLPDARHC | C-D loop of Cε4 | 78 |
| CLHNEVQLPDAC | C-D loop of Cε4 | 80 |

It is envisaged that the mimotopes of the present invention will be of a small size, such that they mimic a region selected from the whole IgE domain in which the native epitope is found. Peptidic mimotopes, therefore, should be less than 100 amino acids in length, preferably shorter than 75 amino acids, more preferably less than 50 amino acids, and most preferable within the range of 4 to 25 amino acids long. Specific examples of preferred peptide mimotopes are P14 and P11, which are respectively 13 and 23 amino acids long. Non-peptidic mimotopes are envisaged to be of a similar size, in terms of molecular volume, to their peptidic counterparts.

It will be apparent to the man skilled in the art which techniques may be used to confirm the status of a specific construct as a mimotope which falls within the scope of the present invention. Such techniques include, but are not restricted to, the following. The putative mimotope can be assayed to ascertain the immunogenicity of the construct, in that antisera raised by the putative mimotope cross-react with the native IgE molecule, and are also functional in blocking allergic mediator release from allergic effector cells. The specificity of these responses can be confirmed by competition experiments by blocking the activity of the antiserum with the mimotope itself or the native IgE, and/or specific monoclonal antibodies that are known to bind the epitope within IgE. Specific examples of such monoclonal antibodies for use in the competition assays include P14/23, P14/31 or P14/33, which would confirm the status of the putative mimotope as a mimotope of P8.

In one embodiment of the present invention at least one IgE epitope or mimotope are linked to carrier molecules to form immunogens for vaccination protocols, preferably wherein the carrier molecules are not related to the native IgE molecule. The mimotopes may be linked via chemical covalent conjugation or by expression of genetically engineered fusion partners, optionally via a linker sequence. As one embodiment, the peptides of the present invention are expressed in a fusion molecule with the fusion partner, wherein the peptide sequence is found within the primary sequence of the fusion partner.

The covalent coupling of the peptide to the immunogenic carrier can be carried out in a manner well known in the art. Thus, for example, for direct covalent coupling it is possible to utilise a carbodiimide, glutaraldehyde or (N-[γ-maleimidobutyryloxy] succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

In a preferred embodiment the present inventors have found that peptides, particularly cyclised peptides may be conjugated to the carrier by preparing Acylhydrazine peptide derivatives.

The peptides/protein carrier constructs can be produced as follows. Acylhydrazine peptide derivatives can be prepared on the solid phase as shown in the following scheme 1 Solid Phase Peptide Synthesis:

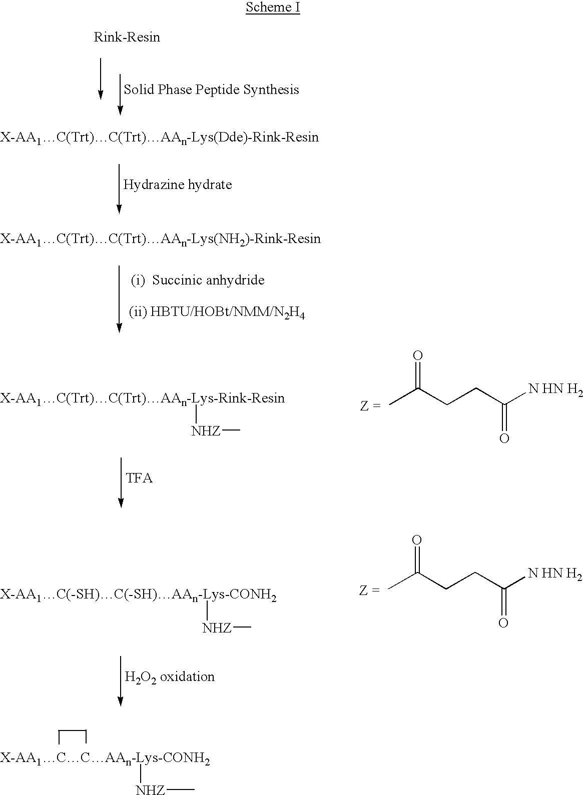

These peptide derivatives can be readily prepared using the well-known ‘Fmoc’ procedure, utilising either polyamide or polyethyleneglycol-polystyrene (PEG-PS) supports in a fully automated apparatus, through techniques well known in the art [techniques and procedures for solid phase synthesis are described in ‘Solid Phase Peptide Synthesis: A Practical Approach’ by E. Atherton and R. C. Sheppard, published by IRL at Oxford University Press (1989)]. Acid mediated cleavage afforded the linear, deprotected, modified peptide. This could be readily oxidised and purified to yield the disulphide-bridged modified epitope using methodology outlined in ‘Methods in Molecular Biology, Vol. 35: Peptide Synthesis Protocols (ed. M. W. Pennington and B. M. Dunn), chapter 7, pp91-171 by D. Andreau et al.

The peptides thus synthesised can then be conjugated to protein carriers using the following technique:

Introduction of the aryl aldehyde functionality utilised the succinimido active ester (BAL-OSu) prepared as shown in scheme 2 (see WO 98/17628 for further details). Substitution of the amino functions of a carrier eg BSA (bovine serum albumin) to ˜50% routinely give soluble modified protein. Greater substitution of the BSA leads to insoluble constructs. BSA and BAL-OSu were mixed in equimolar concentration in DMSO/buffer (see scheme) for 2 hrs. This experimentally derived protocol gives ˜50% substitution of BSA as judged by the Fluorescamine test for free amino groups in the following Scheme 2/3—Modified Carrier Preparation:

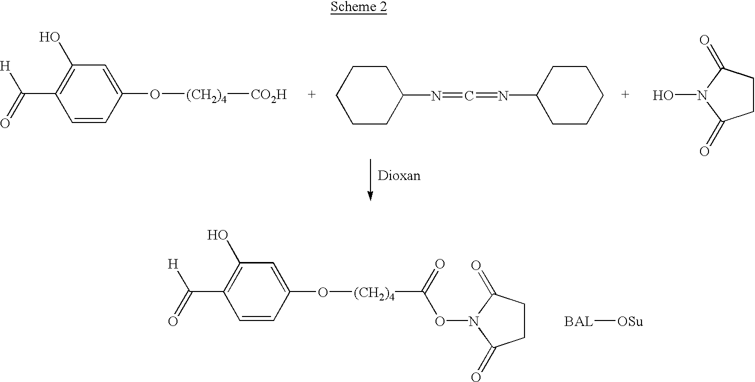

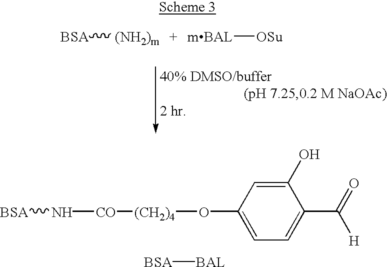

Simple combination of modified peptide and derivatised carrier affords peptide carrier constructs readily isolated by dialysis—Scheme 4—Peptide/carrier conjugate:

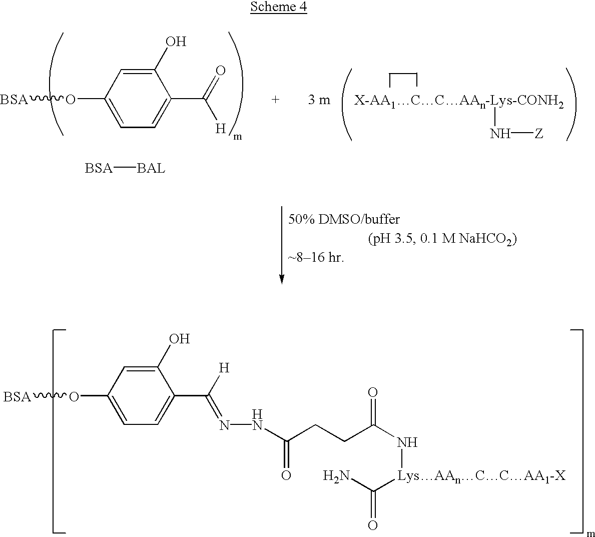

The types of carriers used in the immunogens of the present invention will be readily known to the man skilled in the art. The function of the carrier is to provide cytokine help in order to help induce an immune response against the IgE peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD). Alternatively the mimotopes or epitopes may be directly conjugated to liposome carriers, which may additionally comprise immunogens capable of providing T-cell help. Preferably the ratio of mimotopes to carrier is in the order of 1:1 to 20:1, and preferably each carrier should carry between 3-15 peptides.

In an embodiment of the invention a preferred carrier is Protein D from Haemophilus influenzae (EP0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D ⅓^{rd} (comprising the N-terminal 100-110 amino acids of protein D (GB 9717953.5)).

Another preferred method of presenting the IgE peptides of the present invention is in the context of a recombinant fusion molecule. For example, EP0 421 635 B describes the use of chimaeric hepadnavirus core antigen particles to present foreign peptide sequences in a virus-like particle. As such, immunogens of the present invention may comprise IgE peptides presented in chimaeric particles consisting of hepatitis B core antigen. Additionally, the recombinant fusion proteins may comprise the mimotopes of the present invention and a carrier protein, such as NS1 of the influenza virus. For any recombinantly expressed protein which forms part of the present invention, the nucleic acid which encodes said immunogen also forms an aspect of the present invention.

Peptides used in the present invention can be readily synthesised by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989). Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, N.Y. (1989).

The immunogens of the present invention may comprise the peptides as previously described, including mimotopes or analogues thereof, or may be immunologically cross-reactive derivatives or fragments thereof. Also forming part of the present invention are portions of nucleic acid which encode the immunogens of the present invention or peptides, mimotopes or derivatives thereof.

The present invention, therefore, provides the use of novel epitopes or mimotopes (as defined above) in the manufacture of pharmaceutical compositions for the prophylaxis or therapy of allergies. Immunogens comprising the mimotopes or peptides of the present invention, and carrier molecules are also provided for use in vaccines for the immunoprophylaxis or therapy of allergies. Accordingly, the mimotopes, peptides or immunogens of the present invention are provided for use in medicine, and in the medical treatment or prophylaxis of allergic disease.

Vaccines of the present invention, may advantageously also include an adjuvant. Suitable adjuvants for vaccines of the present invention comprise those adjuvants that are capable of enhancing the antibody responses against the IgE peptide immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (hydroxide or phosphate).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule. Such booster constructs may comprise an alternative carrier or may be in the absence of any carrier.

In a further aspect of the present invention there is provided an immunogen or vaccine as herein described for use in medicine.

The vaccine preparation of the present invention may be used to protect or treat a mammal susceptible to, or suffering from allergies, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. A preferred route of administration is via the transdermal route, for example by skin patches. Accordingly, there is provided a method for the treatment of allergy, comprising the administration of a peptide, immunogen, or ligand of the present invention to a patient who is suffering from or is susceptible to allergy.

The amount of protein in each vaccine dose is selected as an amount which induces an immunoprotective response without significant adverse side effects in typical vaccines. Such amount will vary depending upon which specific immunogen is employed and how it is presented. Generally, it is expected that each dose will comprise 1 -1000 μg of protein, preferably 1-500 μg, more preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

In a related aspect of the present invention are ligands capable of binding to the peptides of the present invention. Example of such ligands are antibodies (or Fab fragments). Also provided are the use of the ligands in medicine, and in the manufacture of medicaments for the treatment of allergies. The term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

Particularly preferred ligands are monoclonal antibodies. For example, P14/23, P14/31 or P14/33 are monoclonal antibodies which recognise P8 (which were raised by vaccination with a P14 immunogen). The hybridomas of these antibodies were deposited as Budapest Treaty patent deposit at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK) on 26 Jan. 2000 under Accession No.s 00012610, 00012611, 00012612 respectively. Also forming an important aspect of the present invention is the use of these monoclonal antibodies in the identification of novel mimotopes of IgE, for subsequent use in allergy therapy, and the use of the antibodies in the manufacture of a medicament for the treatment or prophylaxis of allergy. All of these monoclonal antibodies function in vitro in inhibiting histamine release from human basophils, and also P14/23 and P14/31 have been shown to inhibit passive cutaneous anaphylaxis in vivo.

Therefore, mimotopes of IgE Cε4 that are capable of binding to P14/23, P14/31 or P14/33, and immunogens comprising these mimotopes, form an important aspect of the present invention. Vaccines comprising mimotopes that are capable of binding to P14/23, P14/31 or P14/33 are useful in the treatment of allergy.

Additionally, antibodies induced in one animal by vaccination with the peptides or immunogens of the present invention, may be purified and passively administered to another animal for the prophylaxis or therapy of allergy. The peptides of the present invention may also be used for the generation of monoclonal antibody hybridomas (using know techniques e.g. Köhler and Milstein, Nature, 1975, 256, p495), humanised monoclonal antibodies or CDR grafted monoclonals, by techniques known in the art. Such antibodies may be used in passive immunoprophylaxis or immunotherapy, or be used in the identification of IgE peptide mimotopes.

As the ligands of the present invention may be used for the prophylaxis or treatment of allergy, there is provided pharmaceutical compositions comprising the ligands of the present invention. Preferred pharmaceutical compositions for the treatment or prophylaxis of allergy comprise the monoclonal antibodies P14/23, P14/31 or P14/33.

Aspects of the present invention may also be used in diagnostic assays. For example, panels of ligands which recognise the different peptides of the present invention may be used in assaying titres of anti-IgE present in serum taken from patients. Moreover, the peptides may themselves be used to type the circulating anti-IgE. It may in some circumstances be appropriate to assay circulating anti-IgE levels, for example in atopic patients, and as such the peptides and poly/mono-clonal antibodies of the present invention may be used in the diagnosis of atopy. In addition, the peptides may be used to affinity remove circulating anti-IgE from the blood of patients before re-infusion of the blood back into the patient.

Also forming part of the present invention is a method of identifying peptide immunogens for the immunoprophylaxis or therapy of allergy comprising using a computer model of the structure of IgE, and identifying those peptides of the IgE which are surface exposed. These regions may then be formulated into immunogens and used in medicine. Accordingly, the use of P14/23, P14/31 or P14/33 in the identification of peptides for use in allergy immunoprophylaxis or therapy forms part of the present invention.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4,474,757.

### DESCRIPTION OF DRAWINGS

FIG. 1, Surface exposure of Cε3 an Cε4 of human IgE as calculated from the Padlan and Davis model 1986.

FIG. 2, Histamine release inhibition and anaphylactogenicity of P14 antiserum. Monoclonal Antibodies, PTmAb0005 and PTmAb0011, which were used as positive controls, were added at 1 μg/ml to anti-BSA sera diluted 1/100 and 1/500 (final). The anti-P14 antisera were added at 1/100 and 1/500 final dilution. Cells were taken from an allergic patient sensitive to grass pollen, histamine release was triggered by incubation with this grass pollen allergen.

FIG. 3, Histamine release inhibition and anaphylactogenicity of anti-P14 antiserum. The P14 antiserum from different mice, was added at different dilutions (80× or 40×) to contain approximately 1 μg/ml of anti-IgE antibody as measured by IgE receptor-bound ELISA. Three negative controls were used: Anti-BSA antiserum, non-specific IgG1 and a mixture of non-specific IgG1 diluted in anti-BSA antiserum. mAb11 is a monoclonal antibody known to inhibit histamine release and was used as a positive control (added at 2 μg/ml).

FIG. 4, Histamine release inhibition and anaphylactogenicity of anti-P14 antiserum. Anti-P14 Antisera from different mice were added at a 1/50 final dilution. Monoclonal Abs were added at 2 μg/ml either in assay buffer or in anti-BSA sera dilution 1/50. Three negative controls were used: Anti-BSA antiserum, non-specific IgG1 and a mixture of non-specific IgG1 diluted in anti-BSA antiserum. mAb11 is a monoclonal antibody known to inhibit histamine release and was used as a positive control (added at 2 μg/ml).

FIG. 5, Antibody response anti-P11. Peptide P11 is coated at 1 μg/ml in carbonate buffer at +4° C. overnight. After saturation of plates, two-fold serial dilution of sera are added and incubated for 1h at 37° C. Bound IgG is detected with a biotinylated anti-mouse Ab followed by streptavidin-POD and TMB substrate. Time points measured A. days 14 post vaccination 1, and day 14 post v2; B, Day 14 post v3.

FIG. 6, Anti-P11 IgG anti-human IgE titres. Human IgE was coated at 1 μg/ml. Two-fold serial dilutions of sera (“BSA pool” is a pool of the control group) or PTmAb0005 (a positive control monoclonal antibody) were incubated for 1h at 37° C. Bound IgG is detected with a biotinylated anti-mouse Ab.

FIG. 7, Histamine release inhibition studies with anti-P14 monoclonal antibodies, on allergic basophils donated by dustmite allergic patients (A10 and A11) and from grass pollen allergic patients (G8 and G4). PT11 (PTmAb0011) was used as a positive control, and non-specific IgG2a was used as an isotype control for the P14/23, P14/31 and P14/33.

FIG. 8, IgE domain structure. (A) Each domain is composed of two facing β-sheets, shown in outline, one of 4 anti-parallel β-strands (labelled 4) and the other of 3 anti-parallel β-strands (labelled 3). (B) The seven strands are shown topographically as block arrows labelled a to f, partitioned between the two sheets as shown. The loop-connectivity of the strands is shown topologically with curved arrows: solid arrows are intra-sheet loops and dashed arrows are inter-sheet loops. In the IgG1 Fc domain structures a short c′ strand forms part of the C-D loop, as is predicted for IgE Fc.

FIG. 9, (A) Predicted structural alignment of the A-B loop sequences of human IgE domains Cε2, 3 & 4 with the equivalent segments from the crystallographically determined structure of human IgG1 Fc (domains Cγ2 & Cγ3). β-strands in the IgG1 structure are underlined and labelled a and b; amino acid residues at the ends of each sequence segment are numbered. Vertical arrows below the block of sequences point to predicted optimal cyclisation positions, labelled and connected by dashed or solid lines as shown in FIG. 10b. (B) Predicted structural alignment of the c_d loops of human IgE Cε2,3 & 4 with human IgG1 Fc. β-strands in the IgG1 structure are underlined and labelled c, c′ and d; amino acid residues at the ends of each sequence segment are numbered. Residues highlighted by the shaded boxes form (Cγ2 & Cγ3) or are predicted to form (Cε2, by homology model refinement and experiment, Cε3, Cε4, by homology-modelling) a protected core within the loop. Residues within the plain bold boxes are predicted to be involved in recognition by receptors and/or antibodies. Vertical arrows below the block of sequences point to predicted optimal cyclisation positions, labelled and connected by dashed or solid lines as shown in FIG. 11b.

FIG. 10, (A) The schematic structure of the A-B hairpin at the sheet-sheet interface of Ig constant domains. Adjacent anti-parallel β-strands are shown as solid arrows, labelled a and b. Residues along strand a are labelled i, those along strand b are labelled j. Residues i+n & j+m, where both n and m are zero or even, form part of the sheet-sheet interface within a domain. Residues i+n & j+m, where both n and m are odd, form part of the solvent-exposed surface of a domain. The A-B loop is shown as a black arrow. (B) The schematic structure of the A-B hairpin as in FIG. 3a, with residue positions optimal for cyclisation connected by dashed or solid dumbbells.

FIG. 11, (A) The schematic structure of the C-D hairpin (loop plus supporting β-strands) at the edge of the sheet-sheet interface of Ig constant domains. Opposing anti-parallel β-strands are shown as solid arrows, labelled c and d. Residues along strand c are labelled i, those along strand d are labelled j. Residues i+n & j+m, where n is odd but m is even, form part of the sheet-sheet interface within a domain. Residues i+n & j+m, where n is zero or even but m is odd, form part of the solvent-exposed surface of a domain. The c_d loop, containing the short c′ strand, is shown as a black arrow. (B) The schematic structure of the c_d hairpin, with residue positions optimal for cyclisation connected by dashed or solid dumbbells.

The present invention is illustrated by but not limited to the following examples.

Part 1, Active Vaccination Studies

### EXAMPLES

1.1 Peptide Identification

The peptides were identified by the following technique.

The modelled structure of human IgE has been described Padlan and Davies (Mol. Immunol., 23, 1063-75, 1986). Peptides were identified which were both continuous and solvent exposed. This was achieved by using Molecular Simulations software (MSI) to calculate the accessibility for each IgE amino acid, the accessible surface was averaged over a sliding window of five residues, and thereby identifying regions of the IgE peptides which had an average over that 5-mer of greater than 80 Å².

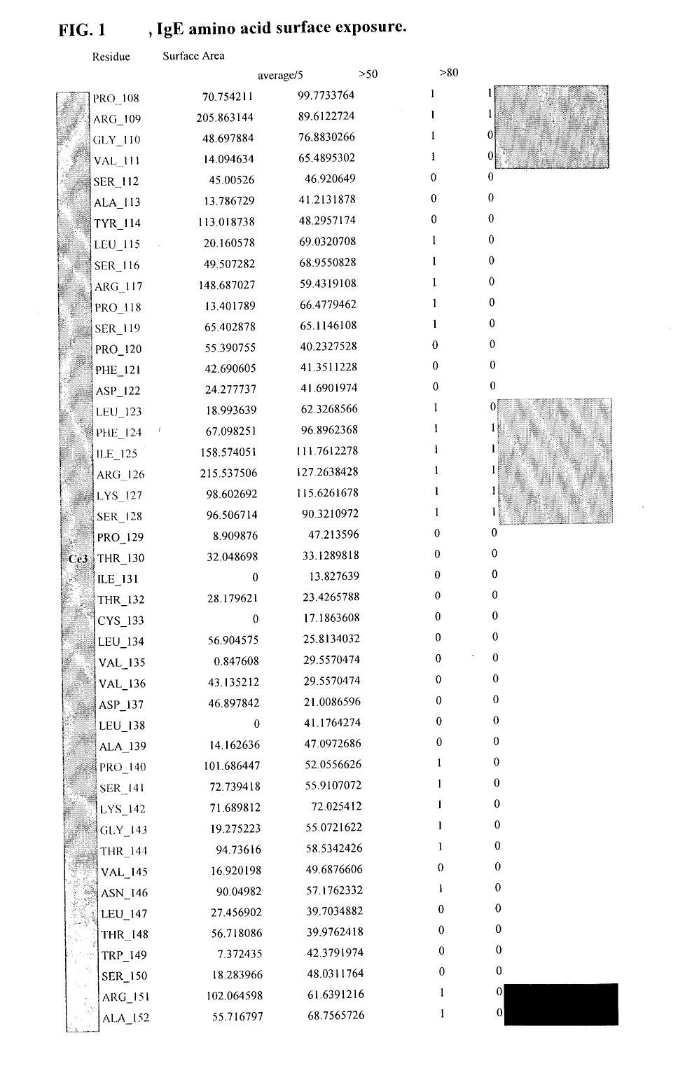

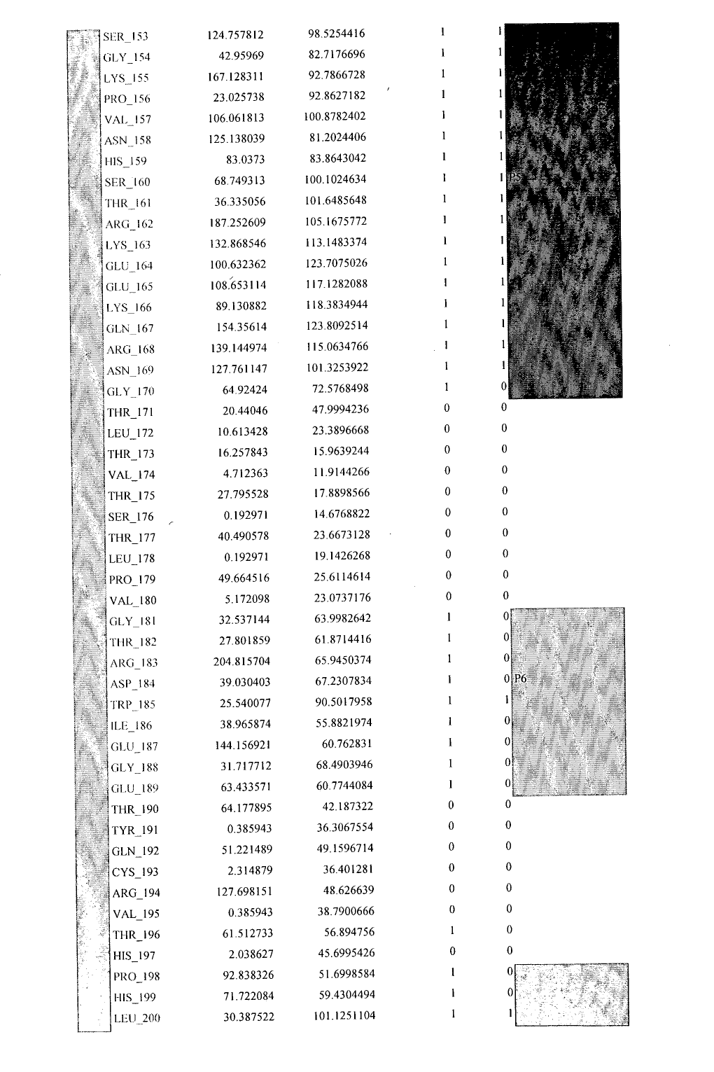

The results of the test are shown in FIG. 1.

Results

From FIG. 1 there are a number of native peptides which may be used as immunogens for raising antibodies against IgE.

TABLE 4
Native surface exposed and continuous IgE peptides
using the 1986 Padlan and Davies model.

| | | Location | SEQ |
|---|---|---|---|
| | | sequence | ID |
| Peptide | Sequence | and IgE Domain | NO. |
| P5 | RASGKPVNHSTRKEEKQRNGTL | Cε3 | 1 |
| P6 | GTRDWIEGE | Cε3 | 2 |
| P7 | PHLPRALMRSTTKTSGPRA | Cε3/Cε4 | 3 |
| P8 | PEWPGSRDKRT | Cε4 (Pro451- | 4 |
| | | Thr461) | |
| P9 | EQKDE | Cε4 | 5 |
| P200 | LSRPSPFDLFIRKSPTITC | Cε3 | 6 |
| P210 | WLHNEVQLPDARHSTTQPRKT | Cε4 | 7 |

In addition to those peptides identified above, the following peptides have been identified using the same selection criteria with the Helm et al. IgE model (2IgE model structure deposited 2/10/90 with PDB (Protein Data Bank, Research Collabarotory for Structural Bioinformatics; http:\pdb-browsers.ebi.ac.uk)).

TABLE 5
Peptides identified using the
Helm et al. 1990 model.

| Name | Sequence | Location | SEQ ID NO. |
|---|---|---|---|
| 1-90N | LFIRKS | Cε3 | 81 |
| 2-90N | PSKGTVN | Cε3 | 82 |
| 3-90N | LHNEVQLPDARHSTTQPRKTKGS | Cε4 | 83 |
| 4-90N | SVNPGK | Cε4 | 84 |

These peptides, or mimotopes thereof, were synthesised and conjugated to carrier proteins for use in immunogenicity studies.

1.2 Synthesis of IgE Peptide/Protein D Conjugates Using a Succinimide-maleimide Cross-linker Protein D may be conjugated directly to IgE peptides to form antigens of the present invention by using a maleimide-succinimide cross-linker. This chemistry allows controlled NH₂ activation of carrier residues by fixing a succinimide group. Maleimide groups is a cysteine-binding site. Therefore, for the purpose of the following examples, the IgE peptides to be conjugated require the addition of an N-terminal cysteine.

The coupling reagent is a selective heterobifunctional cross-linker, one end of the compound activating amino group of the protein carrier by an succinimidyl ester and the other end coupling sulhydryl group of the peptide by a maleimido group. The reactional scheme is as the following:

a. Activation of the protein by reaction between lysine and succinimidyl ester:

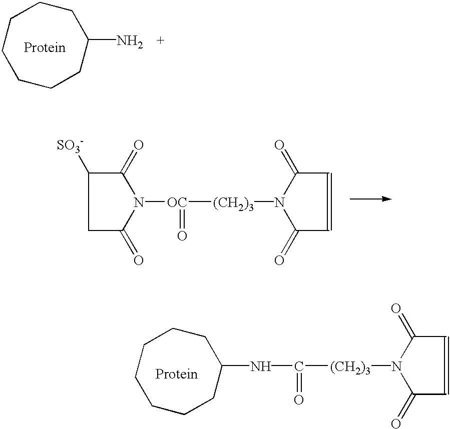

b. Coupling between activated protein and the peptide cysteine by reaction with the maleimido group:

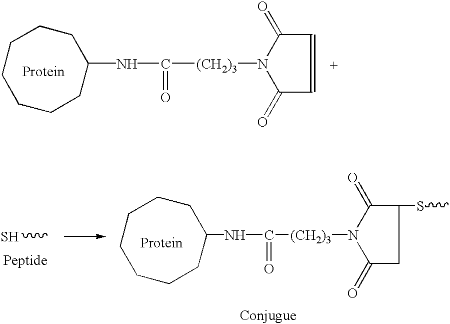

1.3 Preparation of IgE Peptide-Protein D Conjugate

The protein D is dissolved in a phosphate buffer saline at a pH 7.2 at a concentration of 2.5 mg/ml. The coupling reagent (N-[γ-maleimidobutyryloxy] succinimide ester—GMBS) is dissolved at 102.5 mg/ml in DMSO and added to the protein solution. 1.025 mg of GMBS is used for 1 mg of Protein D. The reaction solution is incubated 1 hour at room temperature. The by-products are removed by a desalting step onto a sephacryl 200HR permeation gel. The eluant used is a phosphate buffer saline Tween 80 0.1% pH 6.8. The activated protein is collected and pooled. The peptides (as identified in tables 4 or 5, or derivatives or mimotopes thereof) is dissolved at 4 mg/ml in 0.1 M acetic acid to avoid di-sulfure bond formation. A molar ratio of between 2 to 20 peptides per 1 activated Protein D is used for the coupling. The peptide solution is slowly added to the protein and the mixture is incubated 1 h at 25° C. The pH is kept at a value of 6.6 during the coupling phase. A quenching step is performed by addition of cysteine (0.1 mg cysteine per mg of activated PD dissolved at 4 mg/ml in acetic acid 0.1 M), 30 minutes at 25° C. and a pH of 6.5. Two dialysis against NaCl 150 mM Tween 80 0.1% are performed to remove the excess of cysteine or peptide.

The last step is sterile filtration through a 0.22 μm membrane. The final product is a clear filtrable solution conserved at 4° C. The final ratio of peptide/PD may be determined by amino acid analysis.

In an analogous fashion the peptides of the present invention may be conjugated to other carriers including BSA. A pre-activated BSA may be purchased commercially from Pierce Inc.

Mimotopes of P8 (P14, SEQ ID NO. 20; CLEDGQVMDVDLL) and P5 (P11, SEQ ID NO. 8; CRASGKPVNHSTRKEEKQRNGLL) were synthesised which were conjugated to both Protein D and BSA using techniques described above.

1.4 ELISA Methods

Anti-peptide or Anti-peptide Carrier ELISA

The anti-peptide and anti-carrier immune responses were investigated using an ELISA technique outlined below. Microtiterplates (Nunc) are coated with the specific antigen in PBS (4° overnight) with either: Streptavidin at 2 μg/ml (followed by incubation with biotinylated peptide (1 μM) for 1 hour at 37° C.), Wash 3×PBS-Tween 20 0.1%. Saturate plates with PBS-BSA 1%-Tween 20 0.1% (Sat buffer) for 1 hr at 37°. Add 1° antibody=sera in two-step dilution (in Sat buffer), incubate 1 hr 30 minutes at 37°. Wash 3×. Add 2° anti-mouse Ig (or anti-mouse isotype specific monoclonal antibody) coupled to HRP. Incubate 1 hr at 37°. Wash 5×. Reveal with TMB (BioRad) for 10 minutes at room temperature in the dark. Block reaction with 0.4N H₂SO₄.

Method for the Detection of Anti-Human IgE Reactivity in Mouse Serum (IgE Plate Bound ELISA)

ELISA plates are coated with human chimaeric IgE at 1 μg/ml in pH 9.6 carbonate/bicarbonate coating buffer for 1 hour at 37° C. or overnight at 4° C. Non-specific binding sites are blocked with PBS/0.05% Tween-20 containing 5% w/v Marvel milk powder for 1 hour at 37°° C. Serial dilutions of mouse serum in PBS/0.05% Tween-20/1% w/v BSA/4% New Born Calf serum are then added for 1 hour at 37° C. Polyclonal serum binding is detected with goat anti-mouse IgG-Biotin (1/2000) followed by Streptavidin-HRP(1/1000). Conjugated antibody is detected with TMB substrate at 450 nm. A standard curve of PTmAb0011 is included on each plate so that the anti-IgE reactivity in serum samples can be calculated in μg/ml.

Competition of IgE Binding with Mimotope Peptides, Soluble IgE or PTmAb0011

Single dilutions of polyclonal mouse serum are mixed with single concentrations of either mimotope peptide or human IgE in a pre-blocked polypropylene 96-well plate. Mixtures are incubated for 1 hour at 37° C. and then added to IgE-coated ELISA plates for 1 hour at 37° C. Polyclonal serum binding is detected with goat anti-mouse IgG-Biotin (1/2000) followed by Streptavidin-HRP(1/1000). Conjugated antibody is detected with TMB substrate at 450 nm. For competition between serum and PTmAb0011 for IgE binding, mixtures of serum and PTmAb0011-biotin are added to IgE-coated ELISA plates. PTmAb0011 binding is detected with Streptavidin-HRP(1/1000).

1.5 Human Basophil Assays

Two types of assay were performed with human basophils (HBA), one to determine the anaphylactogenicity of the monoclonal antibodies, consisting of adding the antibodies to isolated PBMC; and a second to measure the inhibition of Lol P I (a strong allergen) triggered histamine release be pre-incubation of the HBA with the monoclonal antibodies.

Blood is collected by venepuncture from allergic donors into tubes containing heparin, and the non-erythrocytic cells were purified. The cells are washed once in HBH/HSA, counted, and re-suspended in HBH/HSA at a cell density of 2.0×10₆ per ml. 100 μl cell suspension are added to wells of a V-bottom 96-well plate containing 100 μl diluted test sample or monoclonal antibody. Each test sample is tested at a range of dilutions with 6 wells for each dilution. Well contents are mixed briefly using a plate shaker, before incubation at 37° C. for 30 minutes.

For each serum dilution 3 wells are triggered by addition of 10 μl Lol p I extract (final dilution 1/10000) and 3 wells have 10 μl HBH/HSA added for assessment of anaphylactogenicity. Well contents are again mixed briefly using a plate shaker, before incubation at 37° C. for a further 30 minutes. Incubations are terminated by centrifugation at 500 g for 5 min. Supernatants are removed for histamine assay using a commercially available histamine EIA measuring kit (Immunotech). Control wells containing cells without test sample are routinely included to determine spontaneous and triggered release. Samples of cells were lysed by 2×freeze/thawing to assay total histamine contained in the cells.

The results are expressed as following:

Anaphylactogenesis Assay

Histamine release due to test samples=% histamine release from test sample treated cells−% spontaneous histamine release.

Blocking Assay

The degree of inhibition of histamine release can be calculated using the formula:

$$\% inhibition = \\ 1 - \frac{( histamine release from test sample treated {cells}^{*} )}{( histamine release from antigen stimulated {cells}^{*} )} \times 100$$

Values corrected for spontaneous release.

### Example 2

### Immunisation of Mice with P14 Conjugates (P14-BSA, P14 -BSA) Induces Production of Anti-human IgE Antibodies

The conjugates comprising the mimotope P14 (25 μg protein/dose), described in example 1, were administered into groups of 10 BalbC mice, adjuvanted with and oil in water emulsion containing QS21 and 3D-MPL described in WO 95/17210. Boosting was be performed on days 14, 24 and 72, sera was harvested 14 days after each immunisation. The immune responses anti-peptide and anti-plate bound IgE was followed using ELISA methods described in Example 1. The antiserum was then tested for anaphylactogenicity and functional activity in the inhibition of histamine release from human allergic basophils (methods as described in example 1).

Immunogenicity Results

Both conjugates, PD-P14 and BSA-P14, were capable of inducing anti-P14 and anti-IgE immune responses. The results for anti peptide and anti-IgE responses, induced by the BSA-P14 conjugates, as measured at day 14 post third and fourth vaccination, are shown in table 6. PTmAb0011 is a monoclonal antibody which is known to bind to the Cε2 domain of IgE, and was used to quantify the anti-IgE responses in μg/ml.

TABLE 6
Immunogenicity results for BSA-P14 conjugates

| | | | Anti-IgE responses | | | | | |
|---|---|---|---|---|---|---|---|---|
| Anti-peptide responses | | | (14 days post 3) | | | Anti-IgE responses (14 | | |
| (14 days post 3) Mid | | | (μg/ml | | | days post 4) (μg/ml | | |
| point titre | | | (PTmAb0011)) | | | (PTmAb0011)) | | |
| AV | SD | GM | AV | SD | GM | AV | SD | GM |
| 25974 | 22667 | 15492 | 9.9 | 2.18 | 0.7 | 22.9 | 33.5 | 4.8 |

Table footnotes:
AV (average),
SD (standard deviation),
GM (geomean)

Mice vaccinated with BSA alone as controls did not generate any detectable anti-peptide or anti-IgE responses.

Functional Activity Results

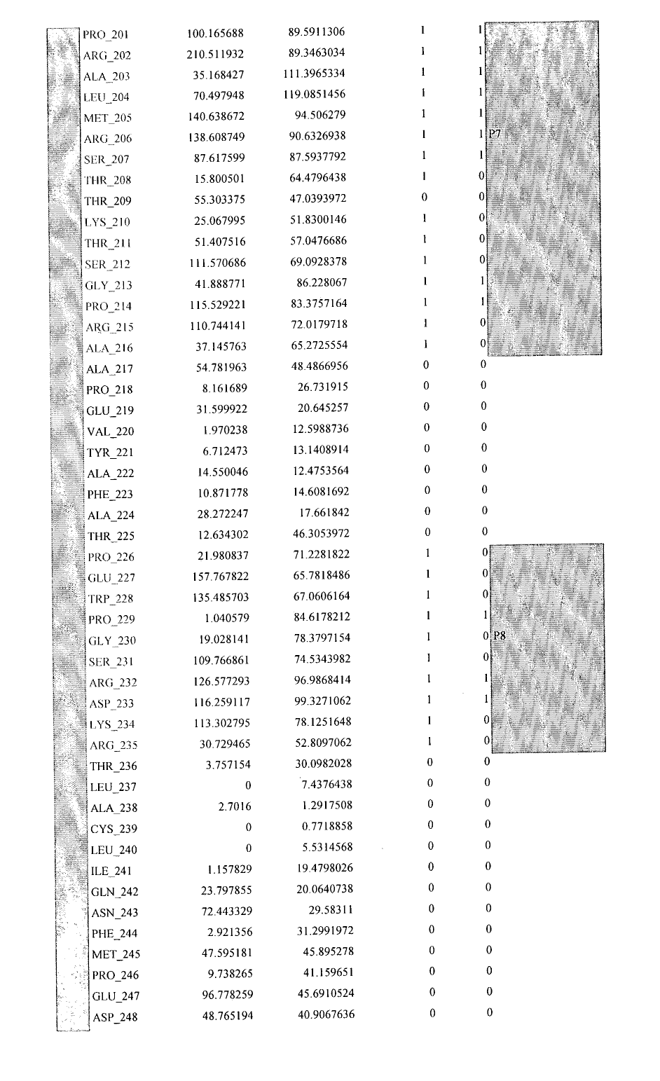

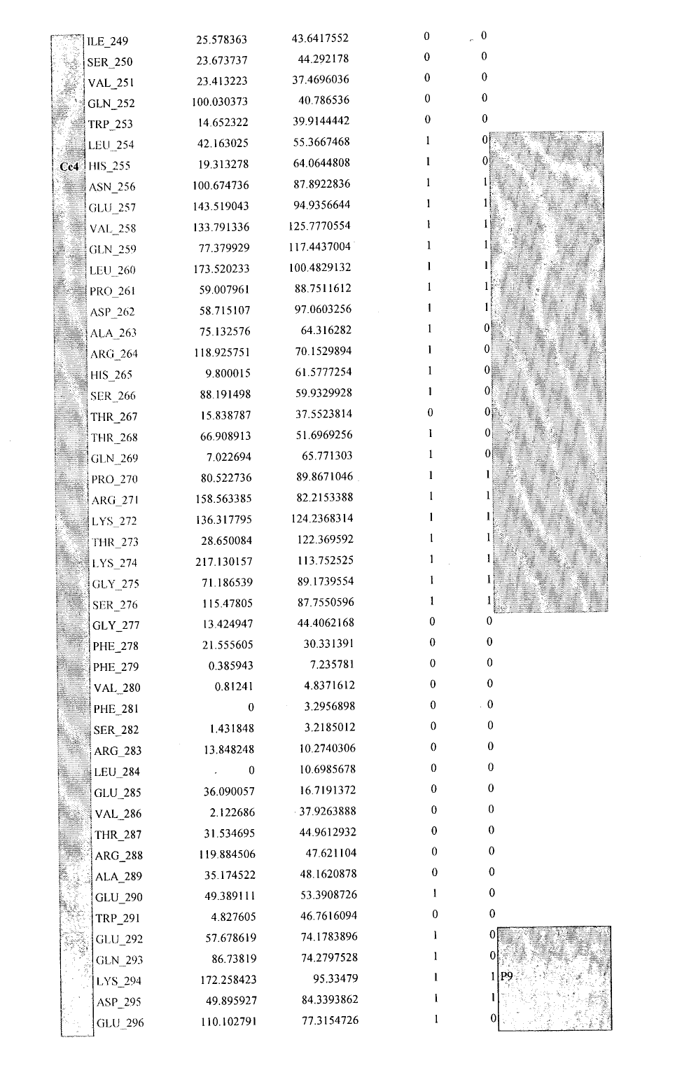

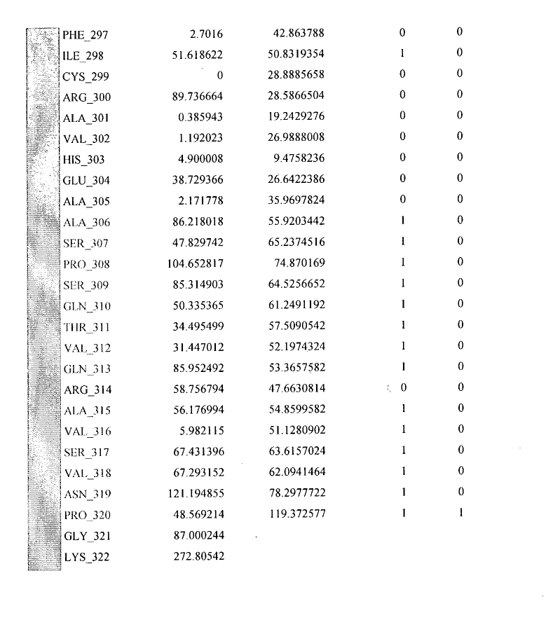

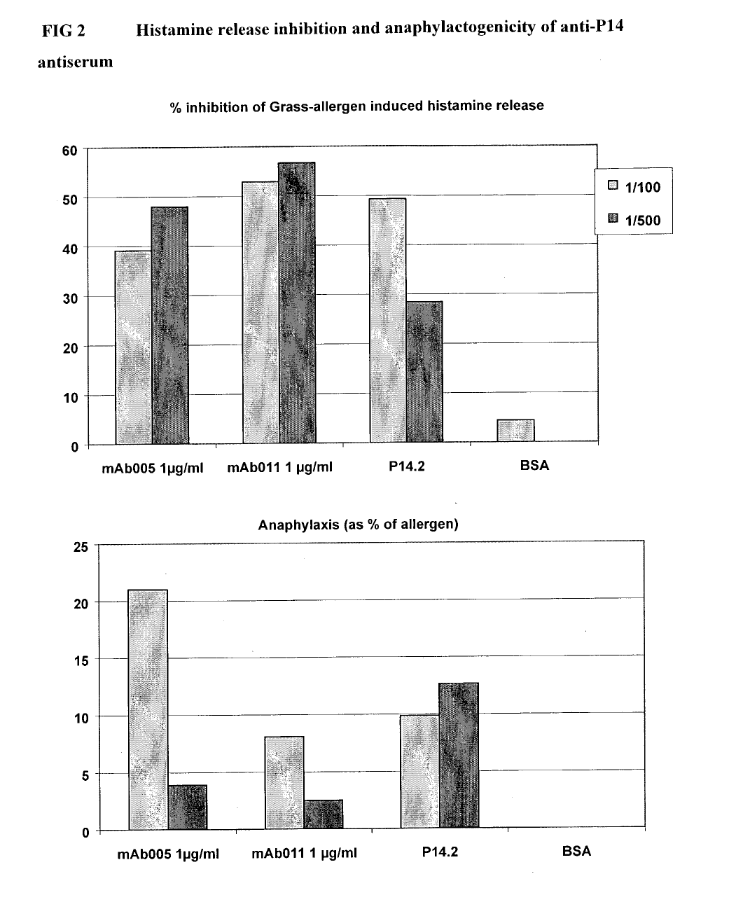

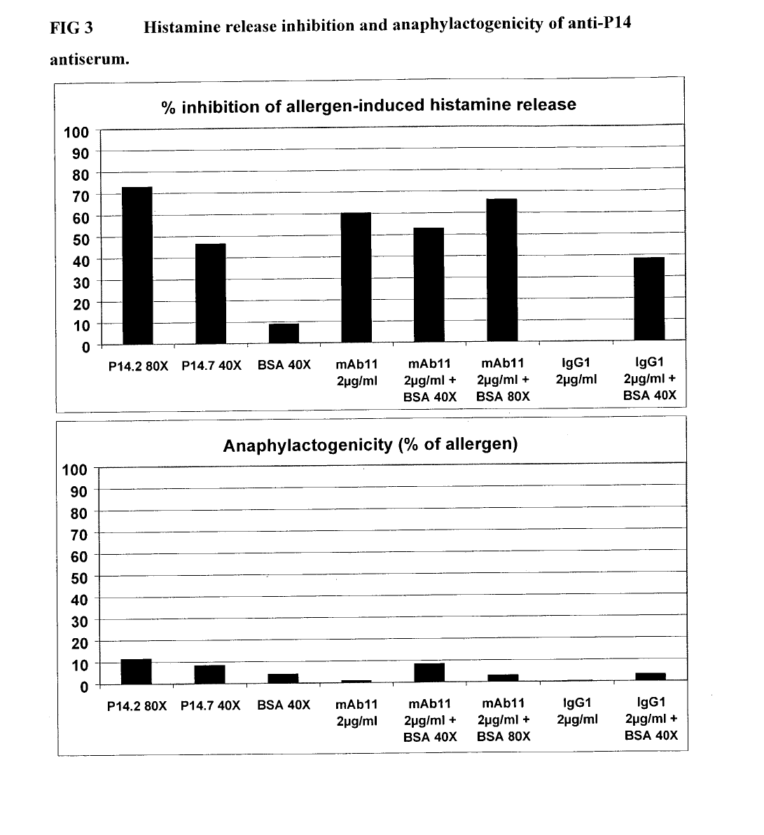

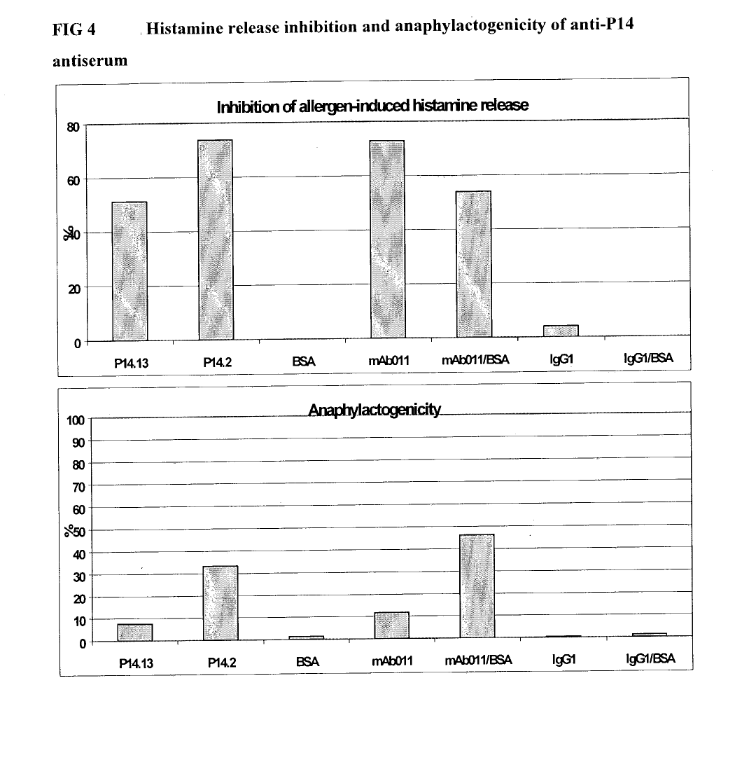

The antiserum raised by the P14 vaccination was found to be functional, in that it was potent in the inhibition of histamine release from allergic human basophils after triggering with allergen (see FIGS. 2, 3 and 4). Moreover, the antiserum was not found to be anaphylactogenic (FIGS. 2, 3 and 4).

Summary

P14 (mimotope of P8) was shown to be capable of raising high titres of anti-P14 and anti-IgE antibodies in mice. These antibodies were subsequently shown to be functional, in that they inhibited histamine release from allergic human basophils, and were not anaphylactogenic. P14 and P8, therefore, may be used in the treatment or prophylaxis of allergy.

### Example 3

### Immunisation of Mice with P11 Conjugates (P11-BSA, P11-BSA) Induces Production of Anti-human IgE Antibodies.

Human IgE epitope peptide P11 was coupled to maleimide-activated BSA (Pierce) (BSA-CRASGKPVNHSTRKEEKQRNGLL). 25 μg of conjugate formulated in SBAS2 was injected IM into 8 female BALB/c mice at days 0, 14 and 28. One control group of mice was injected with BSA/SBAS2. Blood samples were taken 14 days after each injection (a fourth bleeding was performed at day 24 post 3 to increase the availability of sera). Anti-peptide and anti-IgE antibodies raised by vaccination were measured by ELISA, as described in Example 1.

Results

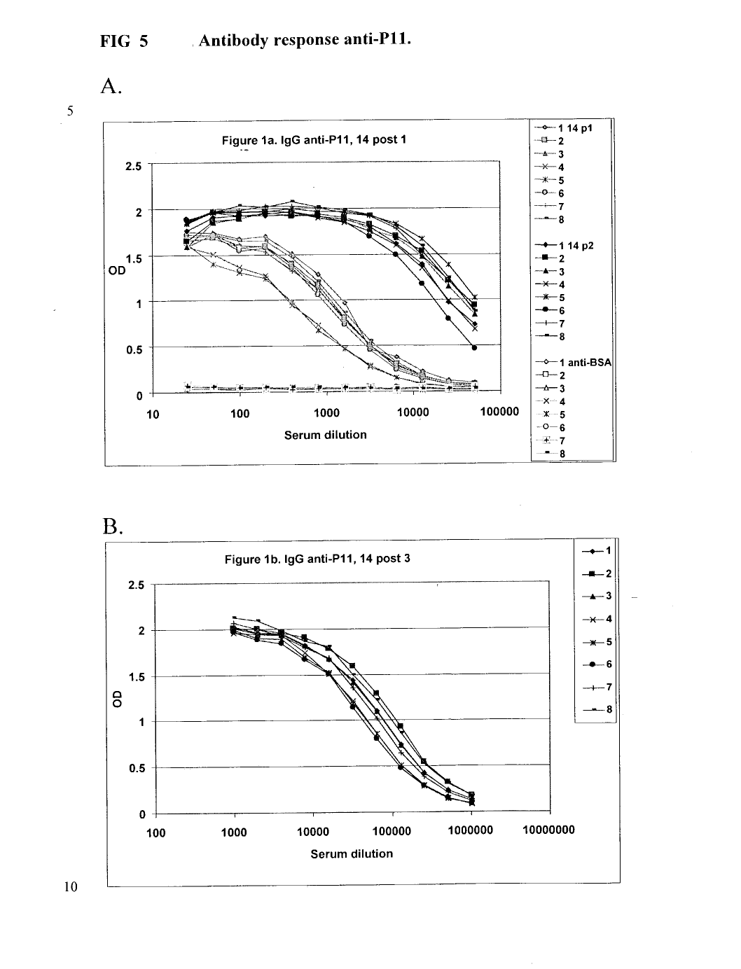

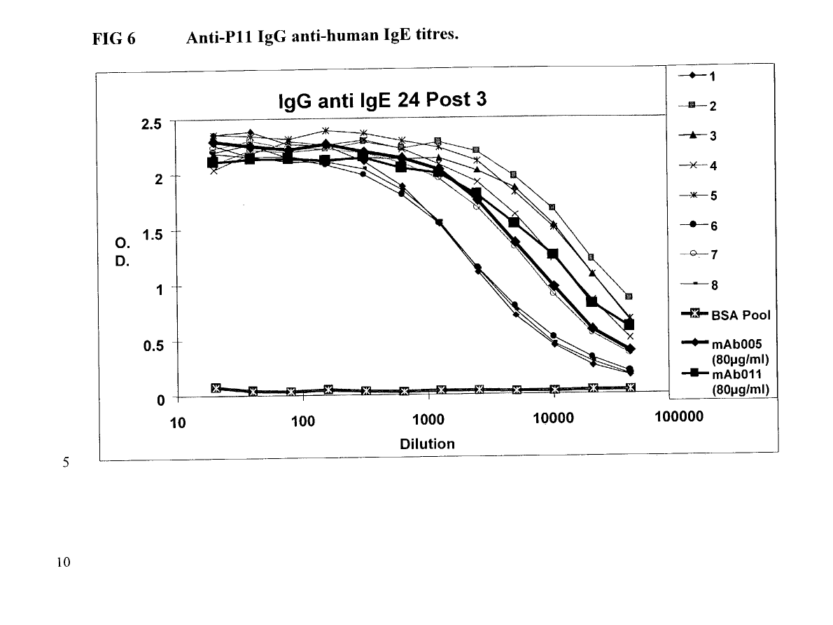

A homogeneous IgG anti-P11 response could be detected already after one injection, but increased further after the second and third injection (FIGS. 5a and 5b). All mice showed an anti-IgE response (ranging from 28-244 μg/ml as expressed in mAb005 equivalents) after a third injection (FIG. 6).

Part 2, Functional Activity of Epitope Specific Monoclonal Antibodies

### Example 4

### Functional Activity of Monoclonal Antibodies Raised Against P14

Monoclonal antibodies have been generated that recognise specifically P8 and mimotopes thereof, using techniques known in the art. Briefly, the P14-BSA conjugate described in part 1 of these examples, was injected into groups of Balb/C mice with the o/w adjuvant containing QS21 and 3D-MPL. Spleen cells were taken and fused with SP2/O B-cell tumour cell line, and supernatants were screened for reactivity against both P14 peptide and IgE. Several cell lines were generated, amongst which were P14/23, P14/31 and P14/33 which were deposited as Budapest Treaty patent deposit at ECACC on 26/1/00 under Accession No.s 00012610, 00012611, 00012612 respectively. All three monoclonal antibodies were confirmed to bind to IgE, and specifically to P14, by ELISA binding assays, and P14 competition assays against monoclonal antibody binding to IgE.

The functional activity of these monoclonal antibodies was assayed in the human basophil histamine release inhibition assay as described in Example 1.

Results

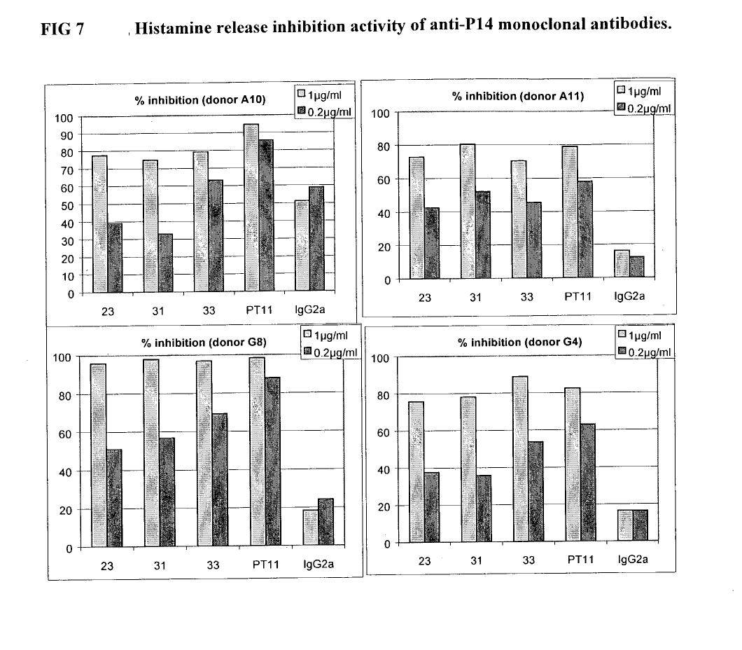

All of the P14 monoclonal antibodies were tested on basophils taken from four different allergic patients (A patients were allergic to dust mite antigen, G patients were allergic to grass pollen). PT11 (PTmAb0011) was included as a positive control antibody which is known to inhibit histamine release in vitro. All of the three P14 monoclonal antibodies (23, 31, and 33) were potent in inhibiting histamine release from allergic basophils (See FIG. 7).

### Example 5

### Anti-IgE Induced in Mice after Immunisation with Conjugate Are Capable of Blocking Local Allergic Response in the Monkey Cutaneous Anaphylaxis Model

P14/23 and P14/31 have also been tested for in vivo activity. Briefly, the local skin mast cells of African green monkeys were shaved and sensitised with intradermal administration of 100 ng of anti-NP IgE (human IgE anti-nitrophenylacetyl (NP) purchased from Serotech) into both arms. After 24 hours, a dose range of the monoclonal antibodies to be tested were injected at the same injection site as the human IgE on one arm. Control sites on the opposite arm of the same animals received either phosphate buffered saline (PBS) or non-specific human IgE (specific for Human Cytomegalovirus (CMV) or Human Immunodeficiency Virus (HIV)). After 5 hours, 10 mg of a BSA-NP conjugate (purchase from Biosearch Laboratories) was administered by intravenous injection. After 15-30 minutes, the control animals develop a readily observable roughly circular oedema from the anyphylaxis, which is measurable in millimeters. Results are expressed in either the mean oedema diameter of groups of three monkeys or as a percentage inhibition in comparison to PBS controls. PTmAb0011, is a monoclonal antibody was used as a positive control. SBmAb0006 was used as a negative control.

TABLE 7
P14/23 results

| Amount of sample | Mean diameter of oedema (mm) | | |
|---|---|---|---|
| to be tested (μg) | P14/23 | mAb0011 | mAb0006 |
| 20 | 0 | ND | 12/15 |
| 10 | 0 | 0 | 17/19 |
| 1 | 15/13 | 0 | 20/20 |
| 0.1 | 15/12 | ND | ND |
| 0.05 | 15/15 | ND | ND |
| 0 | 15/15 | ND | 17/17 |

ND = Not done.

TABLE 8
P14/31 results

| Amount of | | | |
|---|---|---|---|
| sample to | | | |
| be tested | Mean diameter of oedema (mm) | | |
| (μg) | P14/31 | mAb0011 | mAb0006 |
| 20 | 0 | ND | 15/15 |
| 10 | 0 | 0 | 15/15 |
| 1 | 22/25 | 0 | 20/20 |
| 0.1 | 22/25 | ND | ND |
| 0.05 | 25/25 | ND | ND |
| 0 | 20/25 | ND | 20/25 |

As complete inhibition of anaphylaxis was observed with higher doses of monoclonal antibody, these antibodies are not anaphylactogenic per se when administered in vivo.

### Example 6

### Structural Aspects of IgE Mimotopes

The present inventors have shown that the conformation in which the epitopes or mimotopes of the present invention is important for both anti-mimotope antibody recognition, and also for the ability of the peptides to generate a strong anti-IgE immune responses. As such the present inventors have developed structural rules which predict the optimal sites for peptide cyclisation. Peptides that use these sites of cyclisation form one prefered aspect of the present invention.

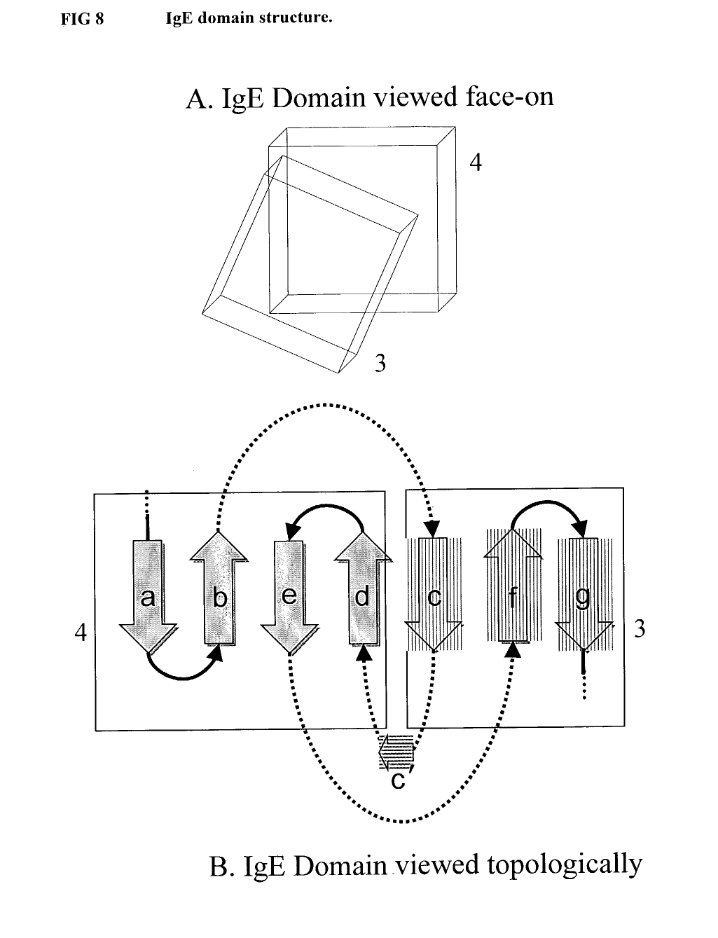

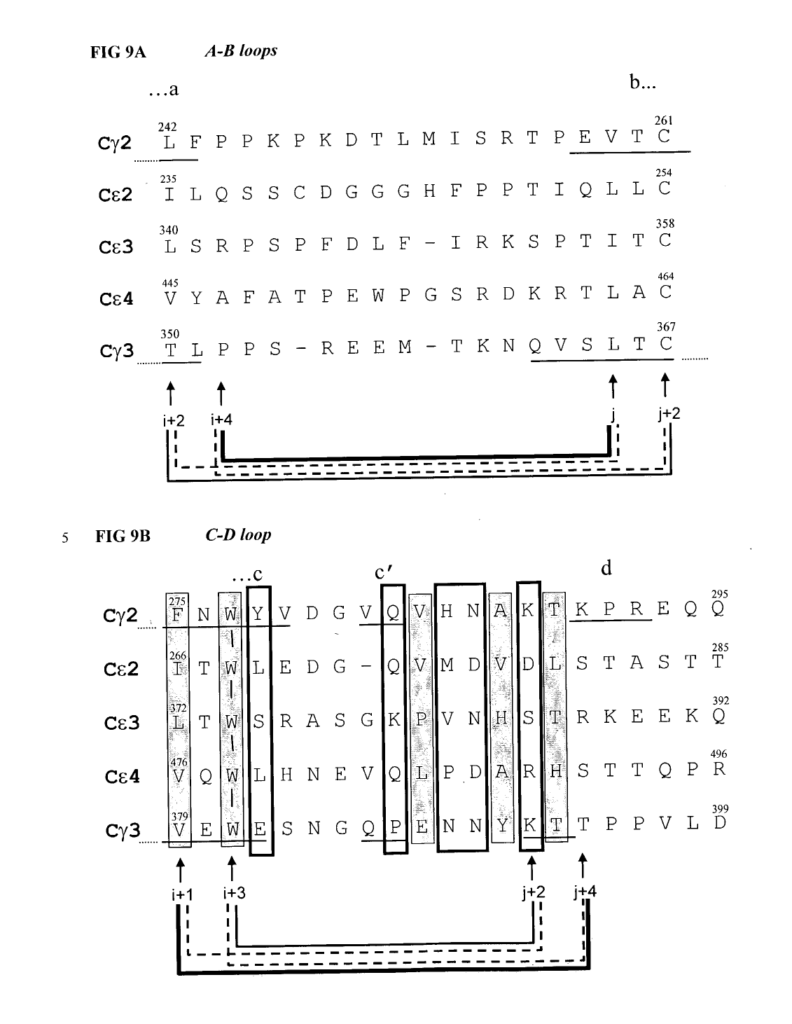

As the full structure of IgE Fc has not been determined, the present inventors have refined the currently available models (Helm et al. supra, Padlan and Davis supra) using the known structure of Cγ2 and Cγ3 of IgG1 (Deisenhofer J., 1981.Biochemistry, 20, 2361-2370). In addition, models of the Cε2 domain have been built by comparison with known Ig folding-unit structures. The present inventors have designed these homology models of IgE Fc and thereby predicted the termini and the gross structure of intra-sheet (A-B loop, FIG. 9A) and inter-sheet loops in IgE Fc domains (C-D loop, FIG. 9B). Having defined the predicted IgE Fc A-B and C-D loops together with their supporting β-strands, mimotopes of the loops may be derived from the wild-type (WT) primary sequence of each loop by covalent cyclisation between chosen specific residues along the adjoining β-strands. Cyclisation is preferably realised by the formation of a disulphide bond between terminal cysteines which therefore combine to become a cystine.

Based upon our structural alignments (FIGS. 9A & 9B) we have derived simple predictive rules in order to enhance the probability that the conformations adopted by a mimotope, after conjugation to a suitable carrier molecule, are similar to those of the parent epitope.

Rule 1

The hydrophobic cystine group should replace WT β-strand residues that belong to the water-inaccessible core of the Ig constant domain, formed by the interface between the two β-sheets.

Rule 2i

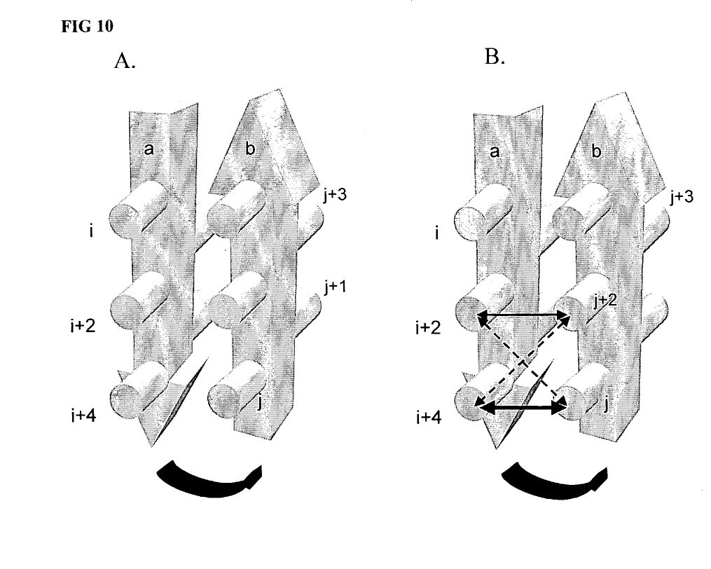

For intra-sheet loops (e.g. the A-B loop) the cystine group should replace WT residues that are from adjacent anti-parallel β-strands (see FIG. 8) and that pack laterally together on the same side of the sheet. Following rule 1, this will be on the domain-interior side of the sheet. The structural derivation of this rule for the A-B loops is shown schematically in FIGS. 10A and 10B.

Rule 2ii

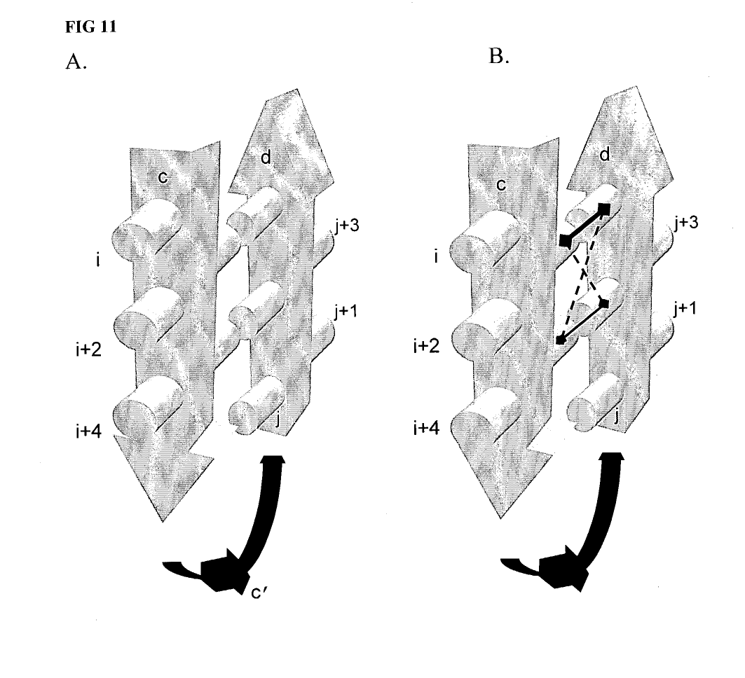

For inter-sheet loops (e.g. the C-D loop) the cystine group should replace WT residues on anti-parallel β-strands, one strand from each sheet. Following rule 1, the residues forming the optimal pair pack together from facing β-sheet surfaces, so forming part of the interface between the sheets. The structural derivation of this rule for the C-D loops is shown schematically in FIG. 11A and FIG. 11B. In the tables of putative mimotope sequences that follow, designs predicted to be optimal are underlined. Below each block of sequences the dotted and solid lines link the residue positions chosen for optimal cyclisation, which are also shown in the same way in FIG. 10B (for A-B loops) and in FIG. 11B (for C-D loops).

Using the sequence alignment as shown in FIGS. 9A and 9B, together with the above rules, the present inventors have designed the following peptides listed in tables 9 to 12. The peptides which are underlined (in solid or dotted lines) are the optimal peptides according to the above identified rules, the same lines are shown in FIG. 10B and FIG. 11B. Non-underlined sequences are mimotopes.

TABLE 9

| | |
|---|---|
| +HZ,1 IgE Cε3 A-B loop sequences | |
| Peptide sequence solid and dotted | |
| underlined are optimal | SEQ ID NO. |

| | |
|---|---|
| 341 357 | 33 |
| C S R P S P F D L F I R K S P T I T C | 34 |
| C S R P S P F D L F I R K S P T I C | 35 |
| C S R P S P F D L F I R K S P T C | 36 |
| C S R P S P F D L F I R K S P C | 37 |
| C S R P S P F D L F I R K S P T C | 38 |
| C S R P S P F D L F I R K S P T I C | 39 |
| C R P S P F D L F I R K S P T I T C | 40 |
| C P S P F D L F I R K S P T C | 41 |
| C P S P F D L F I R K S P T I C | 42 |
| C P S P F D L F I R K S P T C | 43 |
| C P S P F D L F I R K S P C | 44 |

TABLE 10
IgE Cε4 A-B loop sequences

| Peptide sequence (solid and dotted | |
|---|---|
| underlined are optimal) | SEQ ID NO. |
| 446 463 | |
| C Y A F A T P E W P G S R D K R T L A C | 45 |
| C Y A F A T P E W P G S R D K R T L C | 46 |
| C Y A F A T P E W P G S R D K R T C | 47 |
| C Y A F A T P E W P G S R D K R C | 48 |
| C A F A T P E W P G S R D K R C | 49 |
| C A F A T P E W P G S R D K R T C | 50 |
| C A F A T P E W P G S R D K R T L C | 51 |
| C A F A T P E W P G S R D K R T L A C | 52 |
| C F A T P E W P G S R D K R T L A C | 53 |
| C F A T P E W P G S R D K R T L C | 54 |
| C F A T P E W P G S R D K R T C | 55 |
| C F A T P E W P G S R D K R C | 56 |

TABLE 11
IgE Cε3 C-D loop sequences

| Peptide sequence solid and dotted | |
|---|---|
| underlined are optimal | SEQ ID NO. |
| 373 387 | |
| C T W S R A S G K P V N H S T R C | 57 |
| C T W S R A S G K P V N H S T C | 58 |
| C T W S R A S G K P V N H S C | 59 |
| C T W S R A S G K P V N H C | 60 |
| C W S R A S G K P V N H C | 61 |
| C W S R A S G K P V N H S C | 62 |
| C W S R A S G K P V N H S T C | 63 |
| C W S R A S G K P V N H S T R C | 64 |
| C S R A S G K P V N H S T R C | 65 |
| C S R A S G K P V N H S T C | 66 |
| C S R A S G K P V N H S T C | 66 |
| C S R A S G K P V N H S C | 67 |
| C S R A S G K P V N H C | 68 |

TABLE 12
IgE Cε4 C-D loop mimotope sequences

| Peptide sequence (solid and dotted | |
|---|---|
| underlined are optimal) | SEQ ID NO. |
| 477 491 | |
| C Q W L H N E V Q L P D A R H S C | 69 |
| C Q W L H N E V Q L P D A R H C | 70 |
| C Q W L H N E V Q L P D A R C | 71 |
| C Q W L H N E V Q L P D A C | 72 |
| C W L H N E V Q L P D A C | 73 |
| C W L H N E V Q L P D A R C | 74 |
| C W L H N E V Q L P D A R H C | 75 |
| C W L H N E V Q L P D A R H S C | 76 |
| C L H N E V Q L P D A R H S C | 77 |
| C L H E V Q L P D A R H C | 78 |
| C L H N E V Q L P D A R C | 79 |
| C L H N E V Q L P D A C | 80 |

1. A peptide comprising an isolated surface exposed epitope of the Cε3 domain of IgE, wherein the peptide is P5 (SEQ ID No. 1), or mimotope thereof.

2. A peptide comprising an isolated surface exposed epitope of the Cε3 domain of IgE, wherein the peptide is P6 (SEQ ID No. 2), or mimotope thereof.

3. A peptide comprising an isolated surface exposed epitope of the region spanning Cε3 and Cε4 domains of IgE, wherein the peptide is P7 (SEQ ID No. 3), or mimotope thereof.

4. A peptide comprising an isolated surface exposed epitope of the Cε4 domain of IgE, wherein the peptide is P8 (SEQ ID No. 4), or mimotope thereof.

5. A peptide comprising an isolated surface exposed epitope of the Cε4 domain of IgE, wherein the peptide is P9 (SEQ ID No. 5), or mimotope thereof.

6. A peptide comprising an isolated surface exposed epitope of the Cε3 domain of IgE, wherein the peptide is P200 (SEQ ID No. 6), or mimotope thereof.

7. A peptide comprising an isolated surface exposed epitope of the Cε3 domain of IgE, wherein the peptide is P210 (SEQ ID No. 7), or mimotope thereof.

8. A peptide comprising an isolated surface exposed epitope of the Cε3 domain of IgE, wherein the peptide is 2-90N (SEQ ID No. 82), or mimotope thereof.

9. A peptide comprising an isolated surface exposed epitope of the Cε4 domain of IgE, wherein the peptide is 3-90N (SEQ ID No. 83), or mimotope thereof.

10. A peptide comprising an isolated surface exposed epitope of the Cε4 domain of IgE, wherein the peptide is 4-90N (SEQ ID No. 84), or mimotope thereof.

11. A mimotope as claimed in any one of claims 1 to 10 wherein the mimotope is a peptide.

12. A peptide as claimed in claim 4, wherein the mimotope of P8 is a peptide of the general formula:
P, X₁, X₂, P, X₃, X₄, X₅, X₆, X₅, X₅
wherein; X₁ is an amino acid selected from E, D, N, or Q; X₂ is an amino acid selected from W, Y, or F; X₃ is an amino acid selected from G or A, X₄ is an amino acid selected from S, T or M; X₅ is an amino acid selected from R or K; and X₆ is an amino acid selected from D or E.

13. A peptide as claimed in claim 12, wherein the mimotope of P8 is a peptide of the general formula
P, X₁, X₂, P, G, X₄, R, D, X₅, X₅
wherein; X₁ is an amino acid selected from E, D, N, or Q; X₂ is an amino acid selected from W, Y, or F; X₄ is an amino acid selected from S, T or M; X₅ is an amino acid selected from R or K; and X₆ is an amino acid selected from D or E.

14. An immunogen for the treatment of allergy comprising a peptide or mimotope as claimed in any one of claims 1 to 13, additionally comprising a carrier molecule.

15. An immunogen as claimed in claim 14, wherein the carrier molecule is selected from Protein D or Hepatitis B core antigen.

16. An immunogen as claimed in claim 14 or 15, wherein the immunogen is a chemical conjugate of the peptide or mimotope, or wherein the immunogen is expressed as a fusion protein.

17. An immunogen as claimed in any one of claims 14 to 16, wherein the peptide or peptide mimotope is presented within the primary sequence of the carrier.

18. A vaccine for the treatment of allergy comprising an immunogen as claimed in any one of claims 14 to 17, further comprising an adjuvant.

19. A ligand which is capable of recognising the peptides as claimed in any one of claims 1 to 13.

20. A ligand as claimed in claim 19, wherein the ligand is selected from P14/23, P14/31 or P14/33; which are deposited as Budapest Treaty patent deposit at ECACC on 26/1/00 under Accession No.s 00012610, 00012611, 00012612 respectively.

21. A pharmaceutical composition comprising a ligand as claimed in claim 19.

22. A pharmaceutical composition comprising a ligand as claimed in claim 20.

23. A peptide as claimed in any one of claims 1 to 13 for use in medicine.

24. A vaccine as claimed in claim 18 for use in medicine.

25. An immunogen as claimed in any one of claims 14 to 17, for use in medicine.

26. Use of a peptide as claimed in any one of claims 1 to 13 in the manufacture of a medicament for the treatment or prevention of allergy.

27. A ligand which is capable of recognising a peptide as claimed in any one of claims 1 to 13, for use in medicine.

28. Use of a ligand which is capable of recognising a peptide as claimed in any one of claims 1 to 13, in the manufacture of a medicament for the treatment of allergy.

29. Use of P14/23, P14/31 or P14/33; which are deposited as Budapest Treaty patent deposit at ECACC on 26/1/00 under Accession No.s 00012610, 00012611, 00012612 respectively, in the identification of mimotopes of P8.

30. A peptide which is capable of being recognised by P14/23, P14/31 or P14/33; which are deposited as Budapest Treaty patent deposit at ECACC on 26/1/00 under Accession No.s 00012610, 00012611, 00012612 respectively.

31. A vaccine comprising a peptide as claimed in claim 30.

32. A method of manufacturing a vaccine comprising the manufacture of an immunogen as claimed in any one of claims 14 to 17, and formulating the immunogen with an adjuvant.

33. A method for treating a patient suffering from or susceptible to allergy, comprising the administration of a peptide as claimed in any one of claims 1 to 13, to the patient.

34. A method for treating a patient suffering from or susceptible to allergy, comprising the administration of a vaccine as claimed in claim 24 or 31 to the patient.

35. A method of treating a patient suffering from or susceptible to allergy comprising administration of a pharmaceutical composition as claimed in any one of claims 21 or 22, to the patient.
